(19) United States
(12) Patent Application Publication
(10) Pub. No.: US 2024/0268688 A1
(43) Pub. Date: Aug. 15, 2024

(54) METHODS FOR MANUFACTURING SENSORS FOR MEDICAL SYSTEMS AND ASSOCIATED SYSTEMS AND DEVICES

(71) Applicant: Shifamed Holdings, LLC, Campbell, CA (US)

(72) Inventors: Brian Fahey, Menlo Park, CA (US); Peter Andriola, Castro Valley, CA (US); Scott Robertson, San Jose, CA (US); Craig Myers, Hollister, CA (US); Isa Mulvihill, San Francisco, CA (US); Ian Baranowski, Menlo Park, CA (US); David Rahdert, San Francisco, CA (US); Shahn Sage, Clear Lake, MN (US)

(21) Appl. No.: 18/567,561

(22) PCT Filed: Jun. 17, 2022
(86) PCT No.: PCT/US2022/034091

§ 371 Date: Dec. 6, 2023

Related U.S. Application Data
(60) Provisional application No. 63/211,985, filed on Jun. 17, 2021.
(60) Provisional application No. 63/282,660, filed on Nov. 23, 2021.

(51) Int. Cl. A61B 5/0215

(52) U.S. Cl. CPC A61B 5/02158; A61B 2560/04; A61B 2562/12

(57) ABSTRACT

The present technology is generally directed to medical systems having sensors and diaphragms. The sensor device can include a body having a diaphragm. The diaphragm can be formed, for example, by applying a force to the body to create an impression or pattern that corresponds to the diaphragm. The sensor device can be positioned at least partially within a body cavity, and the diaphragm can be configured to flex or bend in response to one or more physiological parameters in the body cavity. In some embodiments, the sensor device can include one or more sensor measurement components configured to measure the one or more physiological parameters based on the flexing or bending of the diaphragm.

### CROSS-REFERENCE TO RELATED APPLICATION(S)

This application claims the benefit of U.S. Provisional Application No. 63/211,985, filed Jun. 17, 2021, and U.S. Provisional Application No. 63/282,660, filed Nov. 23, 2021, both of which are incorporated herein by reference in their entireties.

### TECHNICAL FIELD

The present technology generally relates to implantable medical devices and, in particular, to methods of manufacturing sensors for implantable medical systems and/or devices.

### BACKGROUND

Implantable devices and systems are utilized in modern medicine to provide a host of diagnostic and/or therapeutic benefits. For example, implantable shunting systems are widely used to treat a variety of patient conditions by shunting fluid from a first body region/cavity to a second body region/cavity. The flow of fluid through the shunting systems is primarily controlled by the pressure gradient across the shunt lumen and the geometry (e.g., size) of the shunt lumen. One challenge with conventional shunting systems is selecting the appropriate geometry of the shunt lumen for a particular patient. A lumen that is too small may not provide enough therapy to the patient, while a lumen that is too large may create new issues in the patient. Despite this, most conventional shunts cannot be adjusted once they have been implanted. Accordingly, once the system is implanted, the therapy provided by the shunting system cannot be adjusted or titrated to meet the patient's individual needs.

Some medical systems use sensors to measure physiological parameters (e.g., a shunt device that includes sensors to measure parameters in the first body region and/or the second body region) of the patient, and feedback from the sensors can be utilized to modify or adjust therapy to meet the patient's individual and variable needs.

Conventional medical sensors, and in particular medical sensors intended for implantation via a percutaneous delivery through small sheaths or catheters, are typically costly and difficult to manufacture. More specifically, as components of sensor assemblies become smaller, construction of these sensor assemblies can be associated with numerous challenges. For example, many sensors require isolation between electrical and/or mechanical components that are sensitive to a physiological signal (i.e., the measurement components) and the anatomical environment in which the sensor assembly is implanted (e.g., to prevent fluid ingress that can affect the performance of sensor electronics or other components). Creating a sensor assembly with a housing that acts as a sealed fluid barrier from conventional multi-part constructions can be difficult, and fluid ingress due to leaks, osmosis, or other causes is common over the multi-year operating life of medical sensors. The need for a sensor assembly housing to serve as a fluid barrier between the anatomical environment and the sensor measurement component(s) also creates the need to accurately couple signals from outside of the assembly to the measurement components inside of the sealed housing. This can be done using signal responsive components, coupling elements, and/or other components, which can be particularly difficult to assemble successfully. For example, as sensors get small, some types of sensor assemblies (e.g., pressure sensors) require that the signal-responsive elements that couple signals from the exterior of an assembly housing to the interior of an assembly housing become very thin. Creating a complete and durable fluid seal by affixing these thin components to a housing (e.g., via adhesives, welds, etc.) without damaging the components is difficult, costly, and associated with a high rate of manufacturing failures (i.e., low yield). Thus, there is a need for improved designs for medical sensor housings and improved methods of manufacturing medical sensors.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a schematic illustration of an interatrial device implanted in a heart and configured in accordance with select embodiments of the present technology.

FIG. 2A is a perspective view of a first side of a section of a sensor housing configured in accordance with select embodiments of the present technology.

FIG. 2B is a perspective view of a second side of the section of the sensor housing of FIG. 2A.

FIGS. 2C-2E are partially schematic top and side views of pressure-responsive complexes configured in accordance with embodiments of the present technology.

FIGS. 3A-3C are side cross-sectional views of a sensor housing at various stages of a manufacturing process or method, in accordance with select embodiments of the present technology.

FIG. 4A is a perspective view of a sensor device configured in accordance with select embodiments of the present technology.

FIG. 4B is a side view of the sensor device of FIG. 4A.

FIG. 5 is a perspective view of a section of another sensor housing configured in accordance with embodiments of the present technology.

FIG. 6 is a perspective view of a section of yet another sensor housing configured in accordance with embodiments of the present technology.

FIGS. 7A-7D are perspective views of a barrier component configured in accordance with select embodiments of the present technology.

FIGS. 8A and 8B are perspective views of a barrier component configured in accordance with select embodiments of the present technology.

FIG. 9 is a perspective view of a barrier component configured in accordance with select embodiments of the present technology.

### DETAILED DESCRIPTION

The present technology is generally directed to medical systems having one or more sensor assemblies or devices. In some embodiments, for example, the sensor assembly can include a body having a signal responsive element, such as a pressure responsive diaphragm. The diaphragm can be formed, for example, by applying a force to the body to create an impression or pattern that corresponds to the desired shape/size/geometry of the diaphragm. The diaphragm can include one or more regions that are curved or deformed relative to the body of the sensor assembly. In some embodiments, the diaphragm can be configured to flex or bend in response to one or more physiological parameters (e.g., forces, pressures, etc.) in a body cavity. For example, in some embodiments the sensor assembly can be positioned at least partially in a left atrium and/or a right atrium of a patient's heart, and the diaphragm can be configured to flex or bend in response to a pressure or a change in pressure in the left atrium and/or the right atrium.

The sensor assembly can further include electrical and/or mechanical measurement component(s) that are sensitive to a physiological signal and are configured to measure one or more physiological parameters in an anatomical region, e.g., in response to the flexing or bending of the diaphragm. In some embodiments, for example, a coupling element can be positioned at least partially between the measurement component(s) and the diaphragm, and the coupling element can communicatively couple the measurement component(s) and the diaphragm such that parametric signals that result in the bending or flexing of the diaphragm are conveyed to the measurement component(s). As discussed in further detail below, it is expected that sensor assemblies including such diaphragms configured in accordance with embodiments of the present technology can be manufactured so as to have a number of advantages, including reduced costs, better reliability, improved yields, and/or other advantages.

In some embodiments, the sensor assemblies of the present technology can further include one or more barrier components. Each of the barrier components can be at least partially aligned with one or more of the measurement components of a sensor assembly (e.g., positioned between the measurement component and an environment external to the sensor assembly). Each of the barrier components can be configured to transition between at least a first configuration, in which the barrier component generally conforms to a surface topology or shape of the sensor assembly, and a second configuration in which at least a portion of the barrier component extends away from the sensor assembly, e.g., in a direction generally perpendicular to a longitudinal axis of the sensor assembly. In the second configuration, the barrier component can at least partially prevent tissue growth, fluids, and/or matter from interfering with the operation of the sensor assembly.

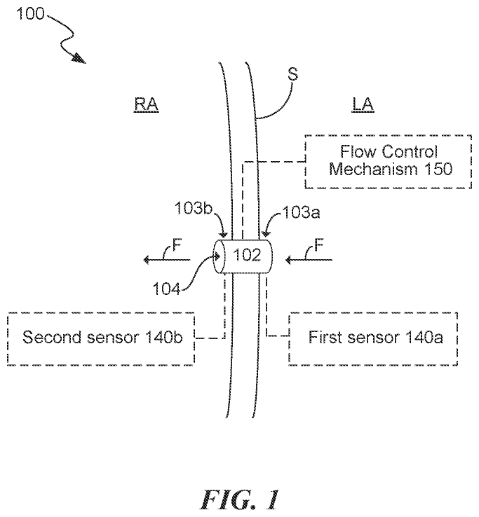

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-9.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” “substantially,” and “about” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a left atrium of a heart) and a second region (e.g., a right atrium or coronary sinus of the heart) of a patient. Although described in terms of a shunt between the atria, namely the left and right atria, one will appreciate that the technology may be applied equally to devices positioned between other chambers and passages of the heart, between other parts of the cardiovascular system, or between other parts of the body. For example, any of the shunts described herein, including those referred to as “interatrial,” may be nevertheless used and/or modified to shunt between the left atrium and the coronary sinus, or between the right pulmonary vein and the superior vena cava. Moreover, while the disclosure herein primarily describes medical devices for shunting blood in the heart, the present technology can be readily adapted for medical devices used to shunt other fluids—for example, devices used for aqueous shunting or cerebrospinal fluid shunting. The present technology may also be adapted to a variety of implanted medical devices in addition to shunts.

The headings provided herein are for convenience only and do not interpret the scope or meaning of the claimed present technology.

Medical Systems with Sensors and Methods of Manufacturing Such Sensors

FIG. 1 is a schematic illustration of an interatrial shunting system 100 (“system 100”) configured in accordance with embodiments of the present technology. The system 100 includes a shunting element 102 defining a lumen 104 therethrough. The shunting element 102 can include a first end portion 103a positioned in a left atrium LA of a patient and a second end portion 103b positioned in a right atrium RA of the patient. Accordingly, when implanted in the septal wall S, the system 100 fluidly connects the left atrium LA and the right atrium RA via the lumen 104. When the system 100 is implanted to treat heart failure, blood generally flows through the lumen 104 in flow direction F (i.e., from the left atrium LA to the right atrium RA).

The system 100 can also include one or more sensor assemblies 140. For example, the system 100 can include a first sensor assembly 140a positionable within the left atrium LA and a second sensor assembly 140b positionable within the right atrium RA. The sensor assemblies 140 can measure one or more physiologic parameters related to the system 100 or the environment proximate to the corresponding sensor assembly 140. For example, the first sensor assembly 140a can be configured to measure left atrium LA pressure and the second sensor assembly 140b can be configured to measure right atrium RA pressure. In some embodiments, the system 100 can further include a processor (not shown) configured to calculate a pressure differential between the left atrium LA and the right atrium RA based on the information measured by the sensor assemblies 140a and 140b. As described below, the system 100 may be adjusted based on the parameters measured by the sensor assemblies 140 and/or the pressure differential calculated by the processor. Additional features of some embodiments of the sensor assemblies 140 useful in the system described herein are disclosed in International (PCT) Application No. PCT/US2020/063360, the disclosure of which is incorporated herein by reference in its entirety.

The system 100 also includes a flow control mechanism 120 (e.g., an actuation mechanism). The flow control mechanism 120 is configured to change a size, shape, or other characteristic of the shunting element 102 to change the flow of fluid through the lumen 104. In some embodiments, the flow control mechanism 120 can selectively change a size and/or shape of the lumen 104 to change the flow through the lumen 104. For example, the flow control mechanism 120 can be configured to selectively increase a diameter of the lumen 104 and/or selectively decrease a diameter of the lumen 104 in response to an input, such as information measured by the sensors 140. In other embodiments, the flow control mechanism 120 is configured to otherwise affect a shape of the lumen 104. Accordingly, the flow control mechanism 120 can be coupled to the shunting element 102 and/or can be included within the shunting element 102. For example, in some embodiments the flow control mechanism 120 is part of the shunting element 102 and at least partially defines the lumen 104. In other embodiments, the flow control mechanism 120 is spaced apart from but operably coupled to the shunting element 102.

In some embodiments, at least a portion of the flow control mechanism 120 can comprise a shape memory material. The shape memory portion can include Nitinol, a shape memory polymer, a pH-based shape memory material, or any other suitable material configured to move or otherwise adjust as would be understood by one of skill from the description herein. The shape memory portion can be characterized by a curve that defines the amount of deformation the portion undergoes in response to a particular input (e.g., an applied stress). For example, the flow control mechanism 120 can include a Nitinol element that is configured to change shape in response to exposure to energy, such as light and/or heat. In such embodiments, the flow control mechanism 250 can be selectively actuated by applying energy directly or indirectly to the Nitinol element. Additional embodiments of flow control mechanisms suitable for use with the present technology are described in International PCT Application No. PCT/US2020/038549, the disclosure of which is incorporated herein by reference in its entirety, and International PCT Application No. PCT/US2020/063360, the disclosure of which was previously incorporated by reference.

In some embodiments, the flow control mechanism 120 is coupled to a processor (not shown) that calculates the pressure differential between the left atrium and right atrium based, at least in part, on the measurements taken by the sensors 140. If the calculated pressure differential falls outside of a predetermined range, the processor can direct the flow control mechanism 120 to change the flow through the shunting element 102.

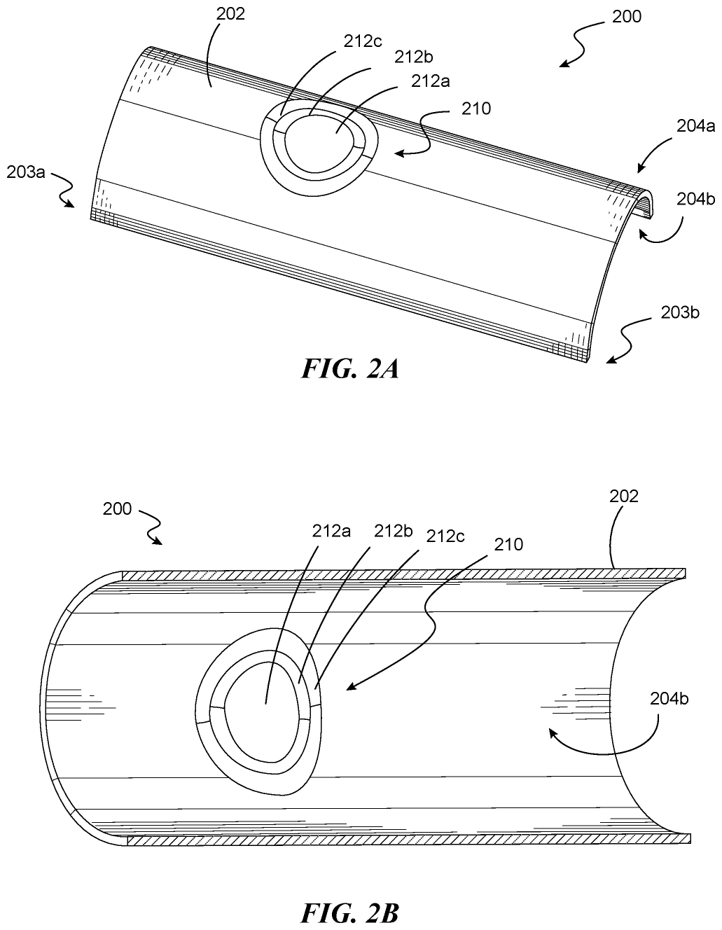

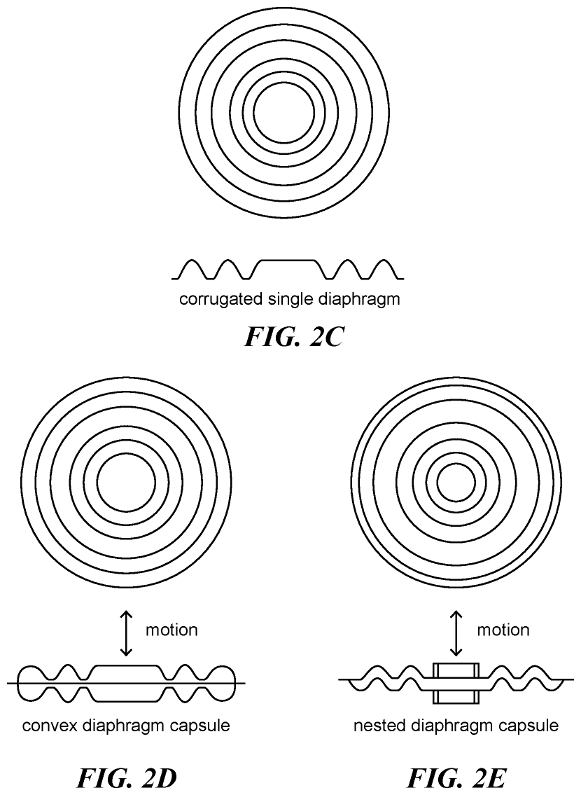

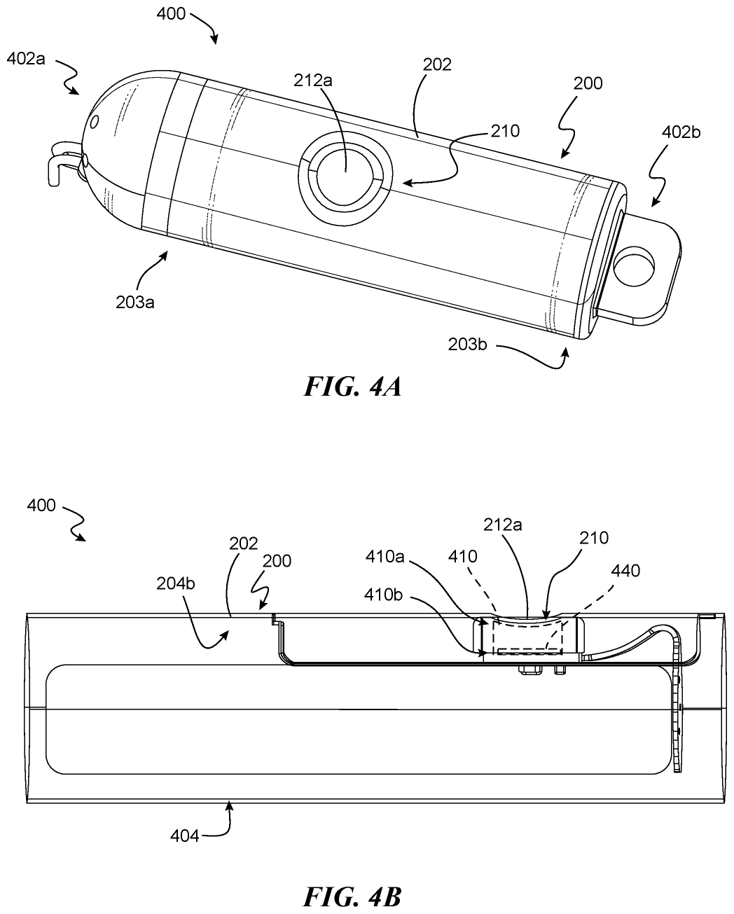

FIGS. 2A and 2B illustrate various views of a section of a sensor housing 200 (“the housing 200”) configured in accordance with embodiments of the present technology. The housing 200 can be part of an interatrial shunting system, such as the system 100 of FIG. 1, or another suitable system configured for implantation within a patient. As described in greater detail below with respect to FIGS. 4A and 4B, for example, the housing 200 can contain one or more measurement components and/or other components associated with a sensor assembly (e.g., the first sensor assembly 140a and/or the second sensor assembly 140b of FIG. 1).

Referring first to FIG. 2A, the housing 200 can include a body 202 having a first end portion 203a and a second end portion 203b. The body 202 includes a first (e.g., exterior) surface 204a and a second, opposite surface 204b. The body 202 can further include a pressure-responsive complex 210 that includes one or more regions 212. In the illustrated embodiment, for example, the pressure-responsive complex 210 includes a membrane or diaphragm 212a and optionally one or more additional regions 212b-c. The diaphragm 212a can be sensitive to and/or configured to flex or bend in response to a force or pressure applied (e.g., a physiological pressure present in an anatomical region) to the first surface 204a of the body 202. In some embodiments, the additional region(s) 212b-c can also be configured to flex or bend in response to a force or pressure applied to the first surface 204a of the body 202. In the illustrated embodiment, for example, the pressure-responsive complex 210 includes a first region featuring the inner diaphragm 212a, a second or intermediate region 212b, and a third or outer region 212c (referred to collectively as “the regions 212”). Each of the regions 212 can be shaped or curved relative to the body 202 (e.g., relative to the first surface 204a and/or the second surface 204b of the body 202). In the illustrated embodiment, for example, the first region (including the diaphragm 212a) has a first (e.g., a substantially flat) curvature, second region 212b has a second curvature, and third region 212c has a third curvature. The first, second, and/or third curvatures can be a same curvature, or one or more of the curvatures can vary. For example, the first, second, and/or third curvatures can be concave, flat, or convex relative to the first surface 204a. In some embodiments, the shape of the second and third regions can help ensure a smooth transition between the body 202 of housing 200 and the diaphragm 212a. In such embodiments, the smooth surface transitions can allow for a predictable healing response once the sensor assembly is implanted in a patient. In some embodiments, the various curvatures of the regions 212 of the pressure-responsive complex 210 can allow for improved sensor sensitivity to a signal by enabling a large, flat diaphragm section to be smoothly transitioned into an otherwise curved surface.

In some embodiments, the thickness of the diaphragm 212a can be 1%, 2% 5%, 10%, 15%, 20% or any other suitable fraction of the thickness of the body 202 of the sensor housing 200. In such embodiments, this allows the diaphragm 212a to have sufficiently low flexural rigidity to transmit external pressure changes through a coupling element (e.g., the coupling element 410 of FIG. 4B) while at the same time allowing body 202 of the sensor housing to have sufficiently high flexural rigidity to retain its intended shape. In some embodiments, the one or more additional regions 212b-c can each have an intermediate thickness between the respective thicknesses of the diaphragm 212a and the body 202 of the sensor housing 200. In some embodiments the thickness of the one or more additional regions 212b-c can be selected to minimize the stress concentration that would otherwise result from an abrupt change in thickness between the diaphragm 212a and the body 202 of the sensor housing 200. Said stress concentration could result in failure at the boundary between the diaphragm 212a and the body 202 of the sensor housing 200.

In general, the shape and size of second, third, and/or additional regions in the pressure-responsive complex 210 can help define the geometry and/or positioning between the diaphragm 212a and the body 202. For example, in some embodiments the housing 200 can be configured to elevate the diaphragm 212a such that it projects outwardly from body 202 (e.g., outwardly from the first surface 204a). This outward projection can have several benefits, such as limiting tissue overgrowth of the diaphragm 212a after the sensor assembly has been implanted and/or to make the tissue overgrowth profile of the diaphragm 212a more predictable. Either of the aforementioned beneficial scenarios can improve sensor functionality, e.g., by limiting or eliminating tissue-response-related sensor drift and/or by making the timeline and/or degree of sensor drift related to tissue-response more standardized or predictable. In some such embodiments, the second and third regions 212b-c can be configured to enable a smooth transition from the body 202 to the level/height of the diaphragm 212a. In variation embodiments, one or more additional regions (e.g., region 212b, region 212c, etc.) can be configured to create an abrupt transition from the body 202, resulting in a button-like projection of the diaphragm 212a that sits in a plateau configuration away from the body 202.

The pressure-responsive complex 210 can have similar or differing processing/treatment finishes and/or surface coating/treatments than the body 202. In some embodiments, for example, the body 202 and the regions 212 of the pressure-responsive complex 210 are all constructed of titanium. In some embodiments, the body 202 can be covered by a surface or otherwise treated to promote rapid tissue overgrowth and/or endothelialization after implantation (e.g., treated to have a sintered, textured, rough, or micro-porous surface, covered in a polyester, ePTFE, or similar material layer, etc.), and the regions 212 can lack these coverings or treatments. In some embodiments, the region(s) 212 can be treated with agents (e.g., pharmaceutical agents) such that tissue overgrowth is eliminated or limited in the region(s) 212, while the body 202 (e.g., portions of the body 202 that do not include the region(s) 212) can be untreated or configured to encourage tissue overgrowth. In embodiments, different regions 212a-c within the pressure-responsive complex 210 can vary in their processing/treatment finish and/or surface coating/treatment. For example, the diaphragm 212a can undergo limited to no post-processing and remain standard machined titanium, while surrounding regions (i.e., region 212b, region 212c, and/or other regions of the body 202) can have at least one surface be processed such that it becomes highly-polished titanium. Without wishing to be bound by theory, it is believed that highly-polished (e.g., electropolished) titanium may create a region of reduced tissue overgrowth that can reduce or prevent endothelial or other tissues from migrating to/adhering to the surface of the diaphragm 212a following implantation of the sensor assembly. Various embodiments of sensor assemblies can be configured to measure different parameters and be manufactured for implantation into different anatomical regions, and thus the processing/treatment finish and/or surface coating/treatments of various components can be adapted to optimize a given sensor assembly for the envisioned implant location and/or measured parameter(s).

FIG. 2B is a perspective view of the second surface 204b of the housing 200. Referring to FIGS. 2A and 2B together, the regions 212 of the pressure-responsive complex 210 on the second surface 204b can have shapes and/or curvature corresponding to the shapes or curvature of the regions 212 on the first surface 204a. In the illustrated embodiment, for example, the third region 212c is concave or inwardly curved relative to the first surface 204a, and the third region 212c is convex or outwardly curved relative to the second surface 204b.

As mentioned previously, the diaphragm 212a can be configured to transmit or communicate one or more physiological parameters (e.g., left atrial pressure, right atrial pressure, etc.) in the body chamber (e.g., left atrium, right atrium, etc.) to the measurement component(s) of a sensor assembly (e.g., the first sensor assembly 140a and/or second sensor assembly 140b of FIG. 1). This can allow the sensor assembly to measure the one or more physiological parameters in the body chamber without the measurement component(s) contacting or being exposed to the environment (e.g., blood, bodily fluids, etc.) of the body chamber. This is described in greater detail below with respect to FIGS. 4A and 4B.

In some embodiments, the pressure-responsive complex 210 can include other arrangements/configuration. FIGS. 2C-2E, for example, illustrate additional configurations for the pressure-responsive complex configured in accordance with embodiments of the present technology. For purposes of illustration and clarity, FIGS. 2C-2E provide a partially schematic top and side view of the pressure-responsive complex without showing the other portions of the housing 200. FIG. 2C, for example, is a partially schematic top and side view of a pressure-responsive complex 210a having a corrugated single diaphragm arrangement. FIG. 2D is a partially schematic top and side view of a pressure-responsive complex 210b having a convex diaphragm capsule arrangement, and FIG. 2E is a partially schematic top and side view of a pressure-responsive complex 210c having a nested diaphragm capsule arrangement. During operation, the diaphragm configurations shown in FIGS. 2C-2E are expected to allow the pressure responsive complex to be responsive to very small pressure differences. The pressure responsive complex configurations disclosed in FIGS. 2C-2E (or in any of the embodiments disclosed herein) can be fabricated using standard machining processes, electrical discharge machining (EDM), or other suitable techniques/processes.

Although the pressure-responsive complex 210 is illustrated as having three regions 212 in FIGS. 2A and 2B, six regions in FIGS. 2C and 2D, and seven regions in FIG. 2E, in other embodiments the pressure-responsive complex 210 can include more or fewer regions 212. For example, in some embodiments the pressure-responsive complex 210 can include one, two, four, five, six, seven, eight, or any suitable number of regions 212. Additionally, although the regions 212 are illustrated as being arranged concentrically relative to each other in FIGS. 2A and 2B, in other embodiments, the regions 212 can be offset from each other, can at least partially overlap, or have any other suitable configuration relative to each other. Additionally, although the diaphragm 212a and the other regions of the pressure-responsive complex 210 are illustrated as having a circular/generally circular shape in FIGS. 2A-2E, in other embodiments the diaphragm and/or one or more of the regions 212 can have a rectilinear, triangular, square, pentagonal, hexagonal, or any other suitable shape.

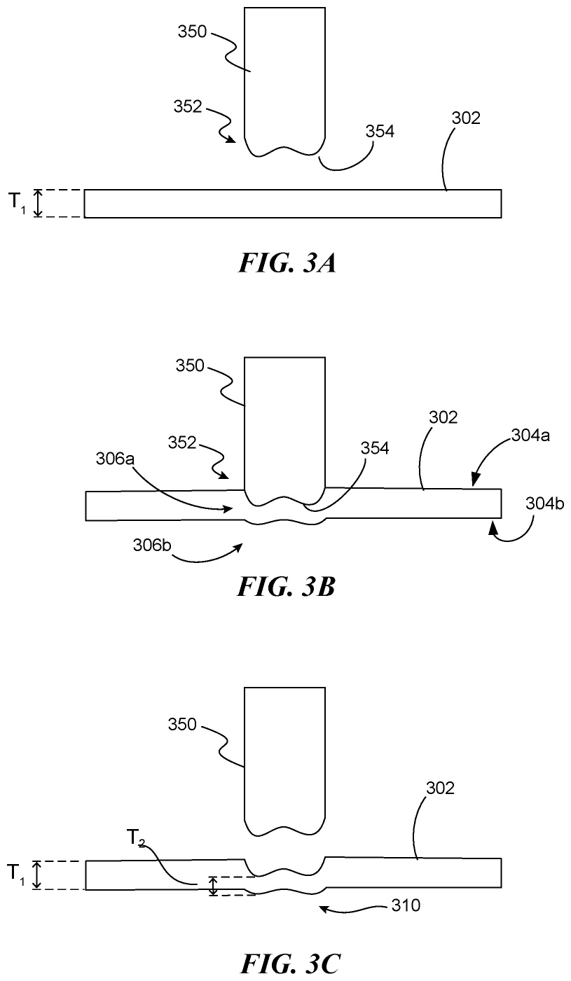

FIGS. 3A-3C are side cross-sectional views of a section of a sensor housing at various stages of a manufacturing process or method, in accordance with embodiments of the present technology. The method can be used to manufacture any embodiment of the sensor housings described herein (e.g., the housing 200 of FIGS. 2A and 2B) and/or one or more components thereof (e.g., the diaphragm 212a of FIGS. 2A and 2B).

Referring first to FIG. 3A, the method includes providing a tool 350 and a substrate 302. The substrate 302 can be generally similar or the same as the body 202 of FIGS. 2A and 2B, and can be used to form a sensor housing, such as the housing 200 of FIGS. 2A and 2B. The substrate 302 can be formed from a metal, such as titanium, and/or any other suitable material. In some embodiments the substrate 302 can be a single sheet of material having a first thickness T₁. The tool 350 can have an end portion 352 that includes a pattern or design 354. The pattern 354 can be configured to correspond to a desired shape/size/thickness/geometry of a pressure-responsive complex and/or diaphragm, such as the pressure-responsive complex 210 and diaphragm 212a of FIGS. 2A and 2B.

Referring next to FIG. 3B, the method includes positioning the end portion 352 of the tool 350 such that the pattern 354 at least partially contacts a first (e.g., outer, upper) surface 304a of the substrate 302 (e.g., a portion of the first surface 304a). In some embodiments, the method includes applying a force to the tool 350 in the direction of the substrate, e.g., such that the pattern 354 produces first deformations 306a in the first surface 304a of the substrate 302 and/or second deformations 306b in a second (e.g., inner, lower) region or surface 304b of the substrate 302. In some embodiments, this can involve cold working or cold pressing processes or techniques such as coining, or hot working processes such as forging, or other such processes known in the art of metal working for plastically deforming a workpiece. In such processes, tool 350 and the associated process parameters such as the force and/or velocity with which the tool 350 is applied to the substrate 302 can be designed for optimal structural properties of each element within the pressure-responsive complex 210 (FIG. 2A), for example creating a ductile, low flexural rigidity diaphragm 212a (FIG. 2A) while work-hardening additional regions 212b-c (FIG. 2A). In some embodiments, the first and/or second deformations 306a-b can be generally similar or the same as the pattern 354 of the tool 350. In some embodiments, the first deformations 306a can correspond to the second deformations 306b, such that if the first deformations 306a are concave relative to the first surface 304a, the second deformations 306b can be convex relative to the second surface 304b.

Referring next to FIG. 3C, the method includes removing the tool 350 from the substrate 302. The first deformations 306a and/or second deformations 306b that remain in the substrate 302 following the removal of tool 350 can form a membrane or diaphragm 310 in the substrate 302, e.g., in the portion of the substrate 302 that was contacted by the tool 350 in FIG. 3B. The diaphragm 310 can be generally similar to or the same as the diaphragm 212a of FIGS. 2A and 2B. The diaphragm 310 can have a second thickness T₂ less than or equal to the first thickness T₁. In some embodiments, the diaphragm 310 can be configured to have a uniform or consistent thickness, such that any portion of the diaphragm has a thickness equal to the second thickness T₂. In some embodiments, the diaphragm 310 can have a variable thickness, and the second thickness T₂ can represent an average thickness of the diaphragm 310. In some embodiments the second thickness T₂ can be at least 0.001 mm, 0.01 mm, 0.25 mm, 0.05 mm, 0.1 mm, 0.25 mm, 0.5 mm, 1 mm, 1.5 mm, 1 mm, or any other suitable dimension. In some embodiments the second thickness T₂ can be at least 1% less than the first thickness T₁, 5% less, 10% less, 20% less, 30% less, 40% less, 50% less, 75% less, 90% less, 95% less, 99% less, or any other suitable percent less than the first thickness T₁. In some embodiments the second thickness T₂ can be configured to correspond to an operating threshold pressure, e.g., such that the diaphragm 310 is pressure sensitive and/or responsive to pressure changes in an environment proximate to the substrate 302. In some embodiments, the method further includes bending or curving the substrate, e.g., to form a body for a sensor housing, such as the body 202 of the sensor housing 200 of FIGS. 2A and 2B.

In some implementations of the methods described herein, the tool 350 or additional tool(s) can be utilized to create other regions of a pressure-responsive complex (e.g., pressure responsive complex 210 as described in connection with FIG. 2) in the substrate 302. For example, a second tool (not shown) can be utilized to create a third region (not shown) in substrate 302 that surrounds the diaphragm 310. This third region can be generally similar to or the same as the region 212b of FIGS. 2A and 2B. This region can have a thickness T₃ that is different from thickness T₁ and thickness T₂, or can have a thickness that is the same as either T₁ and/or T₂. Similarly, this third region can have a shape that differs from the shape of the diaphragm 310 or the surrounding regions of the substrate 302. In one embodiment, this shape formation is accomplished by appropriate design of the pattern 354 at the end portion 352 of the tool 350. In another embodiment, multiple tools, or multiple interfaces with the same tool, can be utilized to create the various sections of a pressure-responsive complex from substrate 302. In still other embodiments, the fabrication/manufacturing process may comprise forming a deformation on only a single side of the substrate 302 (e.g., first surface 304a) without affecting/shaping/modifying the opposing surface (e.g., second surface 304b). In such embodiments, this can involve using EDM or other suitable techniques.

FIGS. 4A and 4B illustrate various views of a sensor assembly/device 400 configured in accordance with embodiments of the present technology. The sensor assembly 400 can be part of an interatrial shunting system, such as the system 100 of FIG. 1, or another suitable medical system configured for implantation within a patient. FIG. 4A, for example, is a perspective view of the sensor assembly 400. The sensor assembly 400 can include the sensor housing 200, the pressure-responsive complex 210, and the diaphragm 212a described in detail above with reference to FIGS. 2A and 2B. The foregoing description of the housing 200, the pressure-responsive complex 210, and diaphragm 212a applies equally to FIGS. 4A and 4B. The sensor assembly 400 can further include a first end portion 402a and a second end portion 402b. The first end portion 402a can be coupled to the first end portion 203a of the body 202, and the second end portion 402b can be coupled to the second end portion 203b of the body 202. In some embodiments, coupling the first and second end portions 402a-b to the respective first and second end portions 203a-b of the body 202 can create or form a substantially fluid-impermeable seal, e.g., to at least partially prevent fluids (e.g., blood) from an anatomical region from entering an interior (FIG. 4B) of the sensor assembly 400. In some embodiments, coupling the first and second end portions 402a-b to the respective first and second end portions 203a-b of the body 202 creates a hermetically-sealed chamber that prevents fluid ingress entirely. In some embodiments, the first and/or second end portions 402a-b are associated with functional aspects of the sensor assembly 400. Within embodiments, the end portions can contain pins/headers for electrical connections, include mechanical features to assist with delivery and/or deployment, include communication components or antennas, or be associated with other features or functionalities.

FIG. 4B is a side view of the sensor assembly 400 where the housing 200 is transparent for the purpose of clarity. As shown, the sensor assembly 400 can include an interior or chamber 404 at least partially defined by the second (e.g., inner) surface 204b of the body 202. In some embodiments, one or more measurement components 440 (shown in dashed line) are positioned in the interior 404 of the device 400. The measurement component(s) 440 can be proximate to the second surface 204b of the body 202 and can be positioned in the region of (e.g., beneath, below, at least partially aligned with, etc.) the diaphragm 212a. The measurement component(s) 440 can be configured to measure one or more physiological parameters in a body region (e.g., a left atrium, a right atrium, etc.). In one embodiment, for example, the measurement component(s) 440 comprise a MEMS sensor configured to measure a pressure (e.g., a left atrial pressure, a right atrial pressure, etc.).

The sensor assembly 400 can further include a coupling element 410 (shown in dashed line) positioned at least partially between the diaphragm 212a and the measurement component(s) 440, as described above. The coupling element 410 can couple (e.g., communicatively couple, operably couple, etc.) the pressure-responsive complex 210 and/or one or more regions thereof (e.g., the diaphragm 212a) and the measurement component(s) 440, such that flexing or bending of the diaphragm 212a is transmitted or communicated to the measurement component(s) 440. For example, the coupling element 410 includes a first (e.g., upper) end portion 410a at least partially contacting the diaphragm 212a, and a second (e.g., lower) end portion 410b at least partially contacting or encapsulating the measurement component(s) 440. In some embodiments, the first end portion 410a can have a geometry that corresponds to the diaphragm 212a, e.g., the diaphragm 212a can have a first geometry including one or more regions (such as the regions 212b-c of FIGS. 2A and 2B), the first end portion 410a can have a second geometry generally similar or the same as the first geometry. In these and other embodiments, the coupling element 410 can be combined with one or more of the pressure responsive complex 210, the diaphragm 212a, and/or the measurement component(s) 440, e.g., to form a single-piece component having part or all of the respective functional and/or geometric aspects/attributes of each of the combined/constituent elements. For example, in an embodiment where the single-piece component includes the coupling element 410 and the diaphragm 212a, the single-piece component can be configured to bend/flex in response to pressure and transmit the pressure to the measurement component(s) 440.

As discussed previously, the diaphragm 212a can be configured to transmit or communicate one or more physiological parameters (e.g., left atrial pressure, right atrial pressure, etc.) in the body chamber (e.g., left atrium, right atrium, etc.) to the measurement component(s) 440. In some embodiments, the bending or flexing of the diaphragm 212a can be transmitted to the measuring component(s) 440 via the coupling element 410, such that the measuring component(s) 440 can measure the forces or pressures in the body chamber. The coupling element 410 can be composed of an elastomeric material such as a silicone elastomer (e.g., polydimethylsiloxane (PDMS)), silicone oil, or another suitable material. In some embodiments, the elastomeric material is initially delivered to the selected region as a liquid and then cured to a solid material. Additional features of some embodiments of coupling elements useful in the systems described herein are disclosed in International Patent App. No. PCT/US2022/034027, filed Jun. 17, 2022, the disclosure of which is incorporated herein by reference in its entirety. In further embodiments, the coupling element 410 can be a fluid.

It is expected that sensor housings including diaphragms configured and/or manufactured in accordance with embodiments of the present technology represent an improvement over traditional sensor housings formed using processes such as cutting, trimming, adhesive or other bonding, or welding/laser welding. For example, in some sensor assembly embodiments it is beneficial to have a very thin (e.g., 1-100 μm) diaphragm. Attaching such a thin diaphragm to a housing in a manner that provides a reliable hermetic seal can be difficult, expensive, and associated with a high rate of manufacturing failures (i.e., low yield). Forming the diaphragm 212a and sensor housing 200 from a single sheet of material as described in the present disclosure are expected to be less costly to produce, be associated with higher manufacturing yields, and be easier to manufacture than such conventional sensor housings. It is additionally expected that forming the diaphragm 212a and sensor housing 200 from a single sheet of material will improve the fluid sealing performance of the sensor assembly 400, thereby reducing or preventing fluids (e.g., blood) from disrupting or interfering with the operation of the one or more measurement components 440. In some embodiments, for example, the operation or normal functioning of the measurement component(s) 440 can be affected or disrupted if they are directly exposed to the environment (e.g., left atrium, blood, bodily fluids, etc.) external to the device 400. Accordingly, providing a sealed housing (and, optionally, a coupling element 410) that at least partially covers or encapsulates the measurement component(s) 440 can isolate the measurement component(s) 440 from the environment external to the device 400. This is expected to reduce or prevent the risk that the functioning or operation of the measurement component(s) 440 and/or related sensor components are negatively affected by the environment external to the sensor assembly 400.

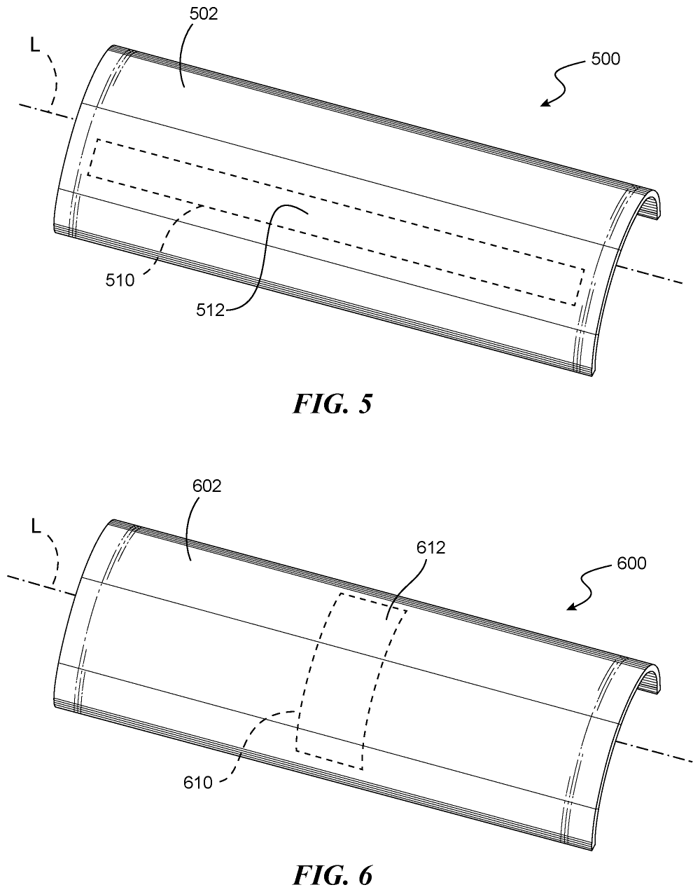

FIGS. 5 and 6 illustrate perspective views of a section of respective sensor housings 500, 600 (“the housings 500, 600”) configured in accordance with additional embodiments of the present technology. The housings 500, 600 can be part of an interatrial shunting system, such as the system 100 of FIG. 1, or another suitable system configured for implantation within a patient. As described previously, for example, the housings 500, 600 can contain one or more measurement components and/or other components associated with a sensor assembly (e.g., the first sensor assembly 140a and/or the second sensor assembly 140b of FIG. 1). Each of the housings 500, 600 can be generally similar to or the same as the housing 200 of FIGS. 2A and 2B. Accordingly, like numbers are used to indicate like components (e.g., pressure-responsive complex 510, 610 versus the pressure responsive complex 210 of FIGS. 2A-2B), and the discussion of the housings 500, 600 will be limited to those features that differ from the housing 200 of FIGS. 2A and 2B or are otherwise provided for context. Additionally, any of the features described with respect to the housings 500, 600 can be combined with one or more features of the housing 200 of FIGS. 2A and 2B, another suitable housing. In at least some embodiments, for example, the housing 200 of the sensor assembly 400 of FIGS. 4A and 4B can include the pressure-responsive complex 510 of FIG. 5 and/or the pressure-responsive complex 610 of FIG. 6.

Referring to FIGS. 5 and 6 together, each of the housings 500, 600 can include a body 502, 602 (“the bodies 502, 602”), respectively, having a corresponding pressure-responsive complex 510, 610 (“the pressure-responsive complexes 510, 610”). The pressure-responsive complexes 510, 610 can include all or part of a circumference and/or a length of the bodies 502, 602. Referring to FIG. 5, for example, the pressure-responsive complex 510 is generally elongate and extends at least partially along the length of the body 502, e.g., generally or substantially aligned with a longitudinal axis L of the body 502 and/or along an axis parallel to the longitudinal axis L. In the embodiment illustrated in FIG. 6, the pressure-responsive complex 610 is generally curved or arcuate and extends at least partially radially around the body 602, e.g., in a direction approximately perpendicular to the longitudinal axis L. In other embodiments, the pressure-responsive complexes 510, 610 can extend along an axis that is angled relative to the longitudinal axis. In further embodiments, the pressure-responsive complexes 510, 610 can extend (e.g., lengthwise, radially, etc.) in a serpentine, zigzag, spiral, helical, and/or any other suitable alignment and/or configuration relative to the longitudinal axis L.

Referring again to FIGS. 5 and 6 together, each of the pressure-responsive complexes 510, 610 can include a corresponding diaphragm 512, 612 (“the diaphragms 512, 612”) generally similar to or the same as the diaphragm 212a of FIGS. 2A and 2B. However, the diaphragms 512, 612 can have a greater thickness than the diaphragm 212a of FIGS. 2A and 2B. In at least some embodiments, for example, the diaphragms 512, 612 and/or the pressure-responsive complexes 510, 610 can have a thickness generally similar to or the same as the thickness of the corresponding body 502, 602. Additionally, in at least some embodiments the pressure-responsive complexes 510, 610 can include a greater surface area of the bodies 502, 602 compared to the pressure responsive complex 210 of FIGS. 2A and 2B. Without being bound by theory, it is believed that housings that include pressure-responsive complexes and/or diaphragms with increased thicknesses and/or surface areas (e.g., such as the pressure-responsive complexes 510, 610 and the diaphragms 512, 612) can have a generally similar or a same (e.g., comparable) sensitivity to forces or pressures as housings that include pressure-responsive complexes and diaphragms with reduced thicknesses and/or surface areas (e.g., such as the pressure-responsive complex 210 and the diaphragm 212a).

Although the pressure-responsive complexes 510, 610 are illustrated as having a rectangular cross-sectional shape in respective FIGS. 5 and 6, in other embodiments each of the pressure-responsive complexes can have a circular shape, a curvilinear shape, a triangular shape, a square shape, a pentagonal shape, a hexagonal shape, a rectilinear shape, or any other suitable shape. Additionally, or alternatively, although the pressure-responsive complexes 510, 610 are illustrated as including specific surface areas of the respective bodies 502, 602, in other embodiments each of the pressure-responsive complexes 510, 610 can have a greater or reduced surface area. In at least some embodiments, for example, each of the pressure-responsive complexes 510, 610 can include between about 5% and about 100% of the surface area of the bodies 502, 602, such as at least 5%, 10%, 20%, 30%, 40%, 50%, 60%, 70%, 80%, 90%, or any other suitable surface area of the bodies 502, 602. Moreover, although the housings 500, 600 in the embodiments illustrated in FIGS. 5 and 6 are both generally cylindrical, it will be appreciated that in other embodiments, the housing 500 and/or the housing 600 may include one or more faces that are rectangular or another suitable shape. Further, while the exterior surfaces of the housing 500, 600 are illustrated as generally smooth, in other embodiments one or more faces of an individual housing may have corrugations, patterns, or other raised elements.

Although each of the housings 500, 600 are illustrated as having a single pressure-responsive complex 510, 610 in FIGS. 5 and 6, in other embodiments each of the housings 500, 600 can include a plurality of pressure-responsive complexes distributed across any suitable portion of the respective housings 500, 600, and individual ones of the plurality of pressure-responsive complexes can have a same or different size, shape, alignment, and/or orientation relative to one or more of other pressure-responsive complexes. In at least some embodiments, for example, the housing 500 and/or the housing 600 can each include one or more of the pressure-responsive complex 510 and one or more of the pressure-responsive complex 610. Further, the housings 500 and 600 may include one or more additional features (e.g., silicone oil, elastomeric material(s) such as PDMS, or other suitable materials) associated with the pressure-responsive complexes 510/610 to further integrate the pressure-responsive complexes with the corresponding sensor assemblies.

As described previously and with reference to FIGS. 2A-3C, each of the housings 500, 600 can be formed from a single sheet of material. Accordingly, it is expected that each of the housings 500, 600 will exhibit at least some or all of the features and improvements described previously and with reference to FIGS. 2A-4B. It is additionally expected that, in some sensor assembly embodiments, it may be advantageous to have sensor housings including pressure-responsive complexes with thicknesses generally similar to or the same as the thicknesses of the bodies of the housings. Sensor assemblies including housings with thickness in accordance with embodiments of the present technology can be more robust and/or less likely to fail due to mechanical and/or other operational forces or stresses.

In other embodiments, other manufacturing methods and processes can be utilized to construct some or all of the embodiments described herein. In at least some embodiments, for example, physical vapor deposition (PVD) and/or any other suitable process known to those of skill in the art can be utilized to construct at least a portion of a housing (e.g., the housing 200 of FIGS. 2A, 2B, 4A, and 4B, the housing 500 of FIG. 5, the housing 600 of FIG. 6, or another housing configured in accordance with the present technology) with a first thickness while leaving another portion of the housing (e.g., a pressure-responsive complex of the housing; such as the pressure-responsive complex 210 of FIGS. 2A, 2B, 4A, and 4B, the pressure-responsive complex 510 of FIG. 5, the pressure-responsive complex 610 of FIG. 6; a diaphragm of the housing, such as the diaphragm 212a of FIGS. 2A, 2B, 4A, and 4B; or another suitable portion of the housing) having a second (e.g., thinner, reduced) thickness. As described previously with respect to FIGS. 2A-3C, 5, and 6, any of the housings described herein can be formed from a single sheet of material, which is expected to increase manufacturing yields due to constructing a sensor assembly from a single component (i.e., removing a step where two components must be attached using welding or other techniques) and also enable a strong fluid-barrier seal that protects an internal section of a sensor assembly from an environment external to the sensor assembly's housing. In these and other embodiments, a pressure-responsive complex (e.g., a diaphragm) can be formed within the housing (e.g., on and/or at least partially within a surface of the housing) using techniques such as laser machining, CNC machining, and EDM, and other suitable techniques. In at least some embodiments, one or more aspects of the sensor assembly (e.g., the pressure responsive complex, the measurement components, etc.) may be manufactured as separate sub-assemblies and subsequently integrated with and/or coupled to at least a portion of the sensor assembly.

Any of the shunting systems and/or sensor assemblies described herein may include one or more additional features to further reduce and/or prevent interference with the operation of these shunting systems and/or sensor assemblies. In some embodiments, for example, it may be desirable or advantageous to isolate or partially isolate one or more portions of a sensor assembly from one or more aspects of the surrounding environment. For instance, many implanted devices can elicit a host response that may result in the growth of tissue “bridges” over, around, and/or otherwise connected to the implanted device. Such tissue bridges may transmit forces from a moving/contracting heart wall to a diaphragm or other section of a pressure responsive complex, which can interfere with the ability of the diaphragm/pressure responsive complex to transmit signals to measurement components that are accurate and reflect pressures in the local environment (e.g., in a heart chamber). Accordingly, it may be desirable or advantageous to limit the growth of these tissue bridges by providing, for example, barriers (e.g., geometric barriers) or other protective features that at least partially block or otherwise prevent tissue growth. However, adding barriers or other protective features to sensor assemblies, such as the sensor assemblies described herein, can be challenging for a number of reasons. First, in many cases it is desirable to configure sensor assemblies for percutaneous/catheter delivery and to have a delivery sheath/catheter be as small as possible to improve the safety of the procedure. For example, delivery of a sensor assembly having a relatively large (e.g., 1 mm or larger) fixed geometric protrusion (e.g., a protrusion extending radially relative to a longitudinal axis of the sensor assembly) may involve the use of a delivery catheter having one or more correspondingly increased dimensions (e.g., an increased diameter) to accommodate the sensor assembly protrusion. The increased size of the delivery catheter can make an associated delivery procedure less attractive to a practitioner and/or a patient, impractical for certain anatomical locations, and/or less safe. Second, as described above, many sensor assembly embodiments require a fluid-impermeable hermetic seal to function and to retain implant safety. As such, “pop-up” or other movable features cannot generally be implemented directly into the sensor assembly housing as doing so can compromise the fluid impermeability of the assembly.

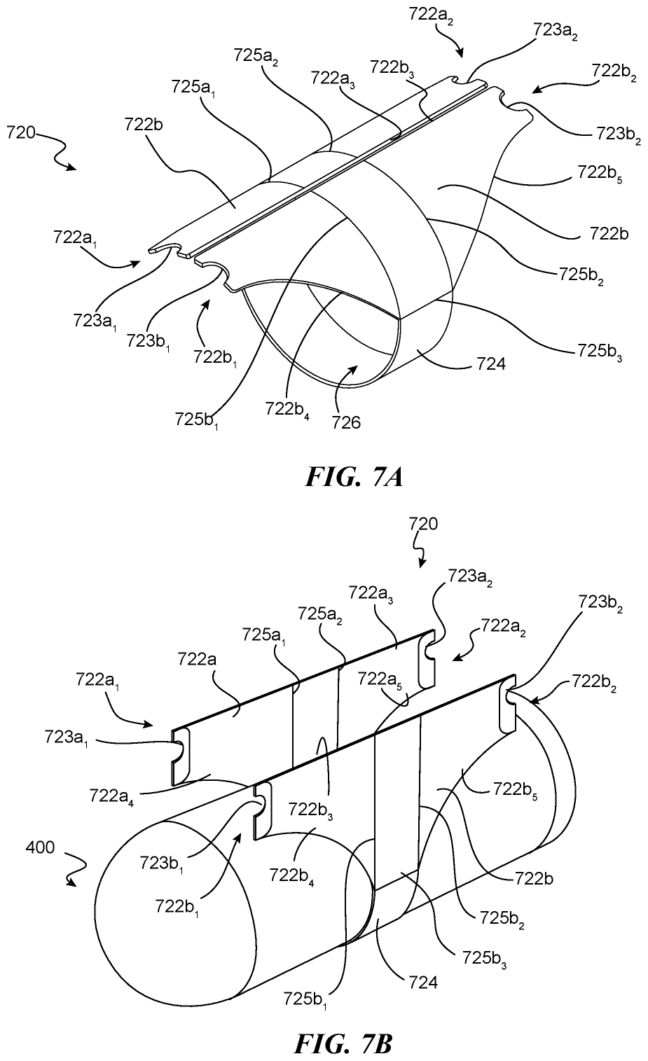

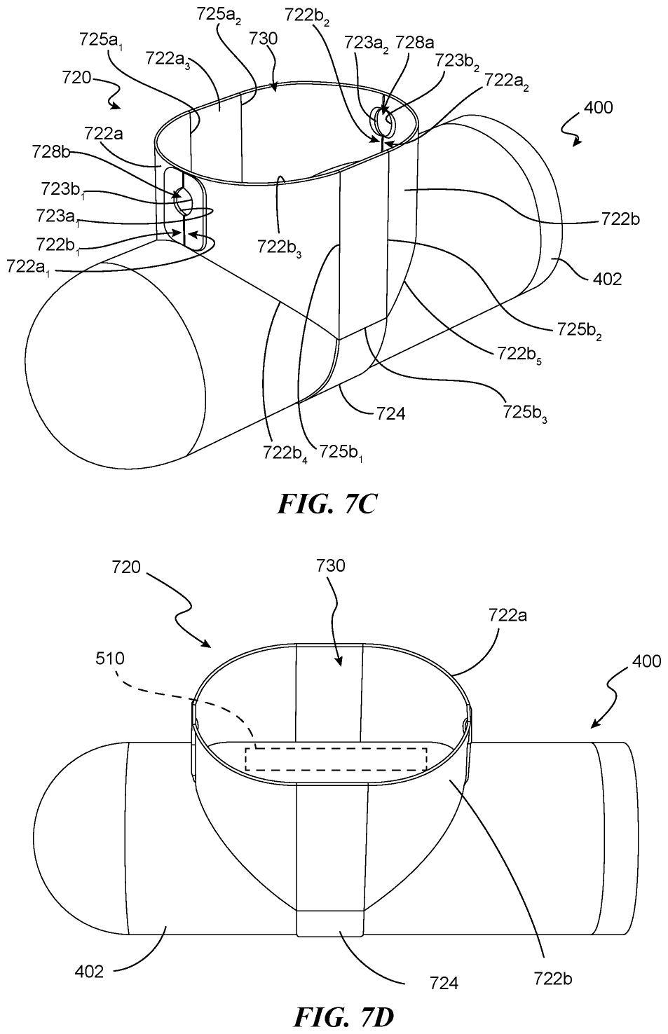

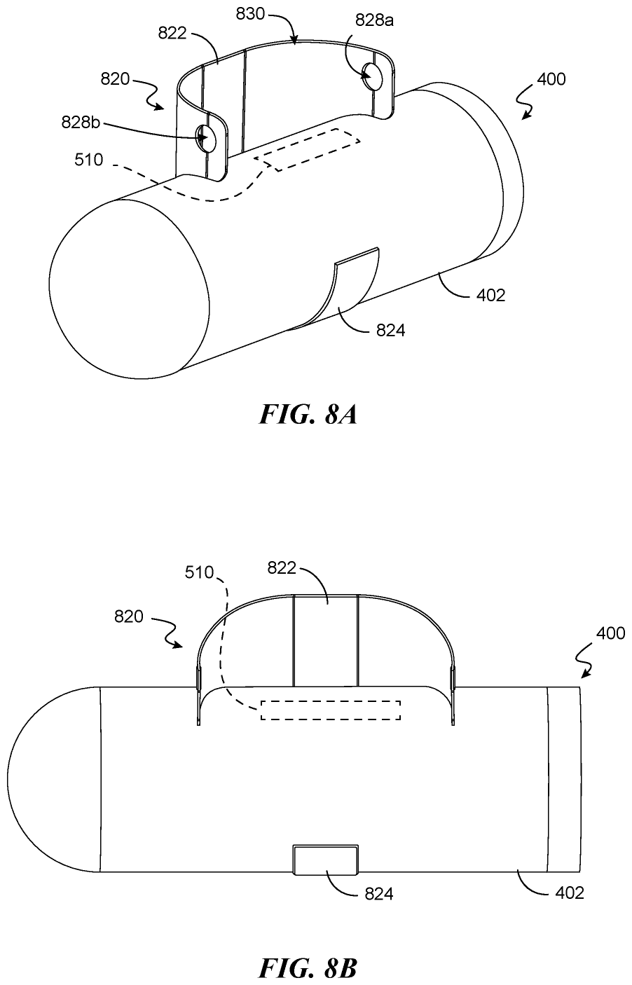

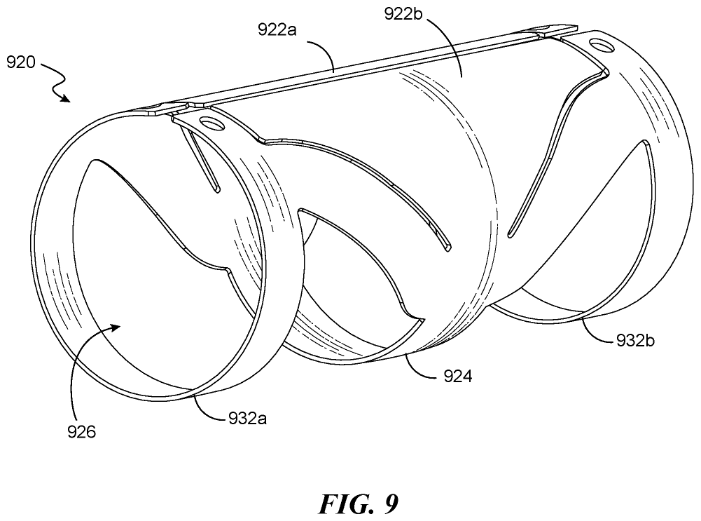

FIGS. 7A-9 illustrate respective protective or barrier components, each configured in accordance with select embodiments of the present technology. Specifically, FIGS. 7A-7D illustrate a barrier component 720, FIGS. 8A and 8B illustrate a barrier component 820, and FIG. 9 illustrates a barrier component 920. The barrier components 720, 820, 920 are each expected to overcome one or more of the challenges described previously, for example, to at least partially prevent tissue growth without or substantially without increasing the size of an associated delivery systems and/or affecting the fluid-impermeable seal of the sensor assembly housing. As described in greater detail below, each of the barrier components 720, 820, 920 can be located proximate to and/or at least partially aligned with the pressure responsive complex and configured to at least partially prevent or otherwise limit host tissue from forming tissues bridges with at least a portion of the pressure responsive complex. Additionally, or alternatively, it is anticipated that the barrier components 720, 820, 920 can act as a strain relief mechanism that at least partially limits the transmittal of forces from native tissues to certain sections of the sensor assembly.

As noted above, FIGS. 7A-7D illustrate barrier component 720. In particular, FIG. 7A is a perspective view of the barrier component 720 in a first (e.g., pre-actuated, undeployed, unexpanded) configuration, FIG. 7B is a perspective view of the barrier component 720 and the sensor assembly 420 with the barrier component 720 in an example intermediate configuration, FIG. 7C is a perspective view of the barrier component 720 in a second (e.g., actuated, deployed, expanded) configuration and the sensor assembly 420, and FIG. 7D is a top perspective view of the barrier component 720 and the sensor assembly 420.

Referring first to FIG. 7A, the barrier component 720 can include a first actuating region 722a, a second actuating region 722b, and an intermediate or support region 724 extending between the first actuating region 722a and the second actuating region 722b. In the illustrated embodiment, the intermediate region 724 is coupled to the first actuating region 722a and the second actuating region 722b at a respective middle or middle portion of the first actuating region 722a and the second actuating region 722b. In other embodiments, the intermediate region 724 can be coupled to the first actuation region 722a and/or the second actuating region 722b at any other suitable portion or position.

The second actuating region 722b can include a first end portion 722b₁, a second end portion 722b₂ opposite the first end portion 722b₁, and a first surface 722b3 extending at least partially between the first end portion 722b₁ and the second end portion 722b₂. Additionally, the second actuating region 722b can include a second surface 722b₄ opposite the first surface 722b₃ and extending at least partially between the first end portion 722b₁ and the intermediate portion 724. The second actuating region 722b can further include a third surface 722b₅ opposite the first surface 722b₃ and/or the second surface 722b₄ and extending at least partially between the second end portion 722b₂ and the intermediate portion 724.

In some embodiments, the first end portion 722b₁ can include a first recess or recessed area 723b₁ and the second end portion 722b₂ can include a second recess or recessed area 723b₂. In the illustrated embodiment, the first recess 723b₁ has a semi-circular shape and the second recess 723b₂ has a size and/or shape corresponding to the size and/or shape of the first recess 723b₁. In other embodiments, however, the first recess 723b₁ and/or the second recess 723b₂ can have different sizes and/or shapes. In these and other embodiments, the first recess 723b₁ and/or the second recess 723b₂ can have a triangular, square, rectangular, rectilinear, curvilinear, or any other suitable shape. In the first configuration, e.g., as illustrated in FIG. 7A, the first surface 722b₃ is generally or substantially linear. As described in greater detail below with respect to FIG. 7C, the configuration of the first surface 722b₃ can be different when the barrier component is in the second configuration (FIG. 7C).

In the illustrated embodiment, the second actuating region 722b is shown to include a plurality of reference lines 725b₁₋₃. The reference lines 725b₁₋₃ are provided for the sake of illustrative clarity, e.g., to illustrate the curvature or contouring of the second actuating region 722b. In at least some embodiments, for example, one or more portions and/or surfaces (e.g., the first end portion 722b₁, the first surface 722b₃, etc.) of the second actuating region 722b may move relative to at least one of the reference lines 725b₁₋₃ when the barrier component transitions between the first configuration (FIG. 7A) and the second configuration (FIG. 7B). It will be appreciated that, in practice, individual ones of the reference lines 725b₁₋₃ may not be visible and/or may have any other suitable positions and/or orientations.

It will be appreciated that, in at least some embodiments, the first actuating region 722a of the barrier component 720 can be configured generally similar to or the same as the second actuating region 722b, with like numbers (e.g., first surface 722a₃ of the first actuating region 722a versus the first surface 722b₃ of the second actuating region 722b) indicating like elements. Accordingly, any description herein of the second actuating region 722b, such as the description above, can apply equally to the first actuating region 722a. In the illustrated embodiment, for example, the first actuating region 722a includes a first surface 722a₃ generally similar to the first surface 722b₃ of the second actuating region 722b; when the barrier component 720 is in the first configuration, the respective first surfaces 722a₃, 722b₃ can be generally or substantially parallel. Additionally, or alternatively, in the first configuration the first and second actuating regions 722a, 722b can each extend in a direction (e.g., a lengthwise direction, between the respective first and second end portions 722a₁₋₂, 722b₁₋₂) generally parallel to a longitudinal axis of the barrier component 720. In these and other embodiments, in the first configuration, the first end portion 722a₁ and the first end portion 722b₁ can each face in a first direction, and the second end portion 722a₂ and the second end portion 722b₂ can each face in a second direction opposite the first direction.

In the illustrated embodiment, the barrier component 720 includes a gap between the first actuating region 722a and the second actuating region 722b, e.g., a gap between the first surface 722a₃ and the first surface 722b₃, when the barrier component 720 is in the first configuration. In other embodiments, the first actuating region 722a and the second actuating region 722b can contact each other (e.g., without or substantially without a gap therebetween the respective first surfaces 722a₃, 722b₃) and/or at least partially overlap each other when the barrier component 720 is in the first configuration.

The barrier component 720 and/or one or more of the regions thereof can be manufactured from Nitinol or any other suitable material that exhibits shape memory, elastic, and/or superelastic properties at body temperature. Accordingly, as described in greater detail below, the barrier component 720 (e.g., the first actuating region 722a and/or the second actuating region 722b) can be actuated to transition the barrier component 720 between the first and second configurations. The barrier component 720 can be manufactured by laser cutting the component from a sheet or tube (e.g., a single sheet or tube) of material, or via any other suitable process or technique known to those in the art.

In operation, the barrier component 720 can be configured to receive and/or be releasably coupled to a sensor assembly, such as the sensor assembly 400 of FIGS. 4A and 4B, or another suitable sensor assembly. In at least some embodiments, for example, the barrier component 720 has a curved or arcuate shape, such that the first actuating region 722a, the second actuating region 722b, and the intermediate region 724 define an opening or aperture 726 (“the opening 726”) of the barrier component 720. The opening 726 can be dimensioned to releasably receive and/or couple to at least a portion of a sensor assembly. In the illustrated embodiment, for example, the opening 726 is dimensioned to receive a housing of a sensor assembly, such as the housing 402 of FIGS. 4A and 4B (e.g., as shown in FIGS. 7B-7D), the housing 500 of FIG. 5, and/or the housing 600 of FIG. 6. In other embodiments, the opening 726 can be dimensioned to receive any other suitable portion of a sensor assembly. The barrier component 720 can be coupled to at least a portion of a sensor assembly via one or more notches, one or more hooks, one or more sutures, an interference fit, a press fit, one or more adhesives, and/or any other suitable coupling process or technique. In some embodiments, the intermediate region 724 is coupled to the sensor assembly, e.g., such that the first and second actuating regions 722a, 722b can be allowed to move relative to the intermediate region 724 to transition the barrier component 720 from the first configuration to the second configuration (FIGS. 7C and 7D). In these and other embodiments, the intermediate region 724 can retain a same shape and/or orientation when the barrier component 720 transitions between the first configuration and the second configuration.

FIG. 7B illustrates an example intermediate configuration of the barrier component 720, e.g., between the first configuration of FIG. 7A and the second configuration of FIG. 7C. It will be appreciated that while FIG. 7B illustrates one suitable intermediate configuration, many other suitable intermediate configurations are also possible. Accordingly, when the barrier component 720 transitions between the first configuration and the second configuration, the barrier component 720 may not assume the intermediate configuration of FIG. 7B and/or may assume a number of other suitable intermediate configurations.

Referring to FIG. 7C, the barrier component 720 can be transitioned from the first configuration (FIG. 7A) to a second configuration (FIG. 7C). In the second configuration, the first end portion 722a₁ of the first actuating region 722a can be at least partially aligned (e.g., contacting) with and/or facing toward the first end portion 722b₁ of the second actuating region 722b. Similarly, the second end portion 722a₂ of the first actuating region 722a can be at least partially aligned with (e.g., contacting) and/or facing toward the second end portion 722b₂ of the second actuating region 722b. Accordingly, in at least some embodiments, transitioning the barrier component 720 from the first configuration (FIG. 7A) to the second configuration (FIG. 7C) can include moving (e.g., inwardly, toward a vertical plane coincident with a longitudinal axis of the barrier component 720) the first end portion 722a₁ of the first actuating region 722a and moving the first end portion 722b₁ of the second actuating region 722b such that the respective first end portions 722a₁, 722b₁ are at least partially aligned and/or facing toward each other. Similarly, transitioning the barrier component 720 from the first configuration (FIG. 7A) to the second configuration (FIG. 7C) can include moving the second end portion 722a₂ of the first actuating region 722a and moving the second end portion 722b₂ of the second actuating region 722b such that the respective second end portions 722a₂, 722b₂ are at least partially aligned and/or facing toward each other. In at least some embodiments, the respective first and second end portions 722a₁₋₂, 722b₁₋₂ of the first and second actuating regions 722a, 722b can move (e.g., all move) in unison. In the second configuration, the alignment of the second end portion 722az with the second end portion 722b₂ can align the second recess 723a₂ of the second end portion 722a₂ with the second recess 723b₂ of the second end portion 722b₂ to form an opening or aperture 728a. Similarly, the alignment of the first end portion 722ai with the first end portion 722b₁ can align the first recess 723a₁ with the first recesses 723a₁ to form another opening or aperture 728b. The shape of each of the openings 728a-b can correspond to the shape of the associated recesses 723a-b (e.g., the opening 728a with the second recesses 723a₂, 723b₂, and the opening 728b with the first recesses 723a₁, 723b₁). Accordingly, in embodiments where each of the recesses 723 have a size and/or shape, each of the openings 728 can have a same size and/or shape.

Additionally, in the second configuration, the first surface 722a₃ of the first actuating region 722a can be generally curved or arcuate and the first surface 722b₃ of the second actuating region 722b can also be generally curved or arcuate. The curvature of each of the first surfaces 722a₃ can correspond to the dimensions of the respective first and second actuating regions 722a, 722b. In embodiments where the first and second actuating regions 722a, 722b have generally similar or the same dimensions, the first surfaces 722a₃, 722b₃ can have generally similar or the same curvature in the second configuration. Transitioning the barrier component 720 from the first configuration (FIG. 7A) to the second configuration (FIG. 7C) can include bending or curving the respective first surfaces 722a₃, 722b₃ of the first and second actuating regions 722a, 722b. In at least some embodiments, the first surfaces 722a₃, 722b₃ can be bent or curved in concert with and/or at least partially in response to the movement of the associated first end portions 722a₁, 722b₁ and/or the second end portions 722a₂, 722b₂ of the respective first and second actuating regions 722a, 722b.

Additionally, in the second configuration, the alignment of the first end portions 722a₁, 722b₁, the alignment of the second end portions 722a₂, 722b₂, and the curvature of the first surfaces 722a₃, 722b₃ can cause the respective first and second actuating regions 722a, 722b of the barrier component 720 to define an outer perimeter or boundary that at least partially surrounds an interior or protected area 730 of the barrier component 720. Thus, the presence of the protected area 730 can be specific or unique to the second configuration of the barrier component 720 (e.g., in the first configuration, the barrier component 720 can lack or otherwise not include the protected area 730). In the illustrated embodiment, the first and second actuating regions 722a, 722b are oriented generally perpendicular to a longitudinal axis of the sensor assembly 400 in the second configuration, e.g., such that the first and second actuating regions 722a, 722b extend away from the sensor assembly 400 to define a wall or protective perimeter around the protected area 730. In other embodiments, the first and second actuating regions 722a, 722b can each have any other suitable orientation relative to the sensor assembly 400, or another suitable sensor assembly. As described in greater detail below regarding FIG. 7D, the protected area 730 can be at least partially aligned with a portion of a sensor assembly, such as a pressure-responsive complex of a sensor assembly, to at least partially block or otherwise prevent tissue growth from interfering with the operation of the portion of the sensor assembly.

In the illustrated embodiment, the barrier component 720 is shown coupled to the sensor assembly 400, e.g., with the sensor assembly 400 positioned at least partially within the opening 726 (FIG. 7A) of the barrier component 720. In the second configuration, one or more of the second surfaces 722a₄, 722b+ and/or the third surfaces 722a₅, 722b₅ of the respective first and second actuating regions 722a, 722b (the second surface 722a₄ and the third surface 722aₛ of the first actuating region 722a are shown in FIG. 7B) can have a contour or shape that is generally similar to or the same as an outer contour or shape of the sensor assembly 400 (e.g., the housing 402 of the sensor assembly 400). In the illustrated embodiment, for example, the housing 402 has a generally circular shape, and each of the second surfaces 722a₄, 722b₄ and the third surfaces 722a₅, 722b₅ has a contour (e.g., curvature, surface topology, etc.) that matches the circular shape of the housing 402). Accordingly, in the second configuration, individual ones of the second surfaces 722a+, 722b+ and/or the third surfaces 722a₅, 722b₅ can at least partially contact the sensor assembly 400. The contact between individual ones of the second surfaces 722a₄, 722b+ and/or the third surfaces 722a₅, 722b₅ with the sensor assembly 400 can assist or aid the intermediate region 724 in coupling the barrier component 720 to the sensor assembly 400 when the barrier component 720 is in the second configuration. In at least some embodiments, for example, the contact between the sensor assembly 400 and individual ones of second surfaces 722a+, 722b+ and/or the third surfaces 722a₅, 722b₅ can help to clamp, secure and/or maintain the alignment/position of the protected area 730 of the barrier component 720 relative to the sensor assembly 400.

FIG. 7D is a top perspective view of the barrier component 720 and the sensor assembly 400 of FIG. 7C. As mentioned previously, the protected area 730 of the barrier component 720 can be at least partially aligned with a portion of the sensor assembly 400 when the barrier component is in the second configuration. In the illustrated embodiment, the sensor assembly 400 includes the pressure-responsive complex 510 of FIG. 5, and the protected area 730 is at least partially aligned with (e.g., at least partially surrounding, containing, barricading, encircling, circumscribing etc.) the pressure-responsive complex 510. Accordingly, in the second configuration, the barrier component 720 can at least partially block or otherwise prevent access to the pressure-responsive complex 510, e.g., to at least partially prevent tissue bridge growth that may interfere with the operation of the pressure-responsive complex 510, as described previously. In operation, for example, the sensor assembly 400 may be positioned proximate a wall of a heart of a subject and, when the barrier component 720 is in the second configuration, the first and/or second actuating regions 722a, 722b can be positioned at least partially between the pressure-responsive complex 510 and the heart wall and at least prevent tissue from growing between the heart wall and the pressure-responsive complex 510. In other embodiments, the protected area 730 can be aligned with another pressure-responsive complex and/or any other suitable portion of the sensor assembly 400, or another sensor assembly. Additionally, the openings 728a-b (FIG. 7C) defined by the first and second actuating regions 722a, 722b can allow blood and other fluids to flow in and/or out of the protected area 730, such that any fluid within the protected area 730 can flow out of the protected area 730 via one or more of the openings 728a-b. Accordingly, the openings 728a-b are expected to reduce the likelihood of the protected area 730 being filled with blood and/or other fluids, e.g., that may interfere with the operation of the pressure-responsive complex 510. Additionally, or alternatively, it is anticipated that in some scenarios the barrier component 720 can act as a strain relief mechanism that at least partially limits the transmittal of forces from native tissues to certain sections of the sensor assembly. In the illustrated embodiment, for example, the orientation of the first and second actuating regions 722a, 722b can at least partially prevent tissues (e.g., host tissues) in the external environment from contacting the pressure-responsive complex 510, which can reduce the likelihood of host tissue acting directly on the pressure-responsive complex 510 and, in turn, can improve the accuracy of the sensor assembly 400.

Referring to FIGS. 7A-7D together, the barrier component 720 can be constructed such that each of the actuating regions 722a, 722b can change its geometry/orientation between the first configuration (FIG. 7A) and the second configuration (FIGS. 7C and 7D). In some embodiments (e.g., those where barrier component 720 is comprised at least in part of Nitinol that is manufactured such that its austenite finish (A_{f}) temperature is lower than or comparable to body temperature), the geometry of each of the actuating regions 722a, 722b can be shape-set into a larger deployed configuration (e.g., the second configuration shown in FIGS. 7C and 7D) during manufacturing, and subsequently be manipulated into a more compact delivery configuration (e.g., the first configuration shown in FIG. 7A) and loaded into a delivery tool such as a sheath or catheter for insertion/implantation into a subject. When contained within a delivery system such as a sheath or catheter, forces from the boundaries of the delivery system can maintain each of the actuating regions 722a, 722b in the compact delivery (first) configuration. When the sensor assembly is deployed from a delivery sheath or catheter into a body and the forces applied on each of the actuating regions 722a, 722b by the delivery system are removed, the superelastic properties of barrier component 720 cause each of the actuating regions 722a, 722b to automatically actuate and regain their respective shape set configurations, e.g., automatically transitioning the barrier component 720 from the first configuration (FIG. 7A) to the second configuration (FIGS. 7C and 7D). As described previously, when in the expanded deployed state (e.g., the second configuration), each of the actuating regions 722a, 722b can create a fence-like barrier that isolates all or a portion of the sensor assembly (e.g., the pressure responsive complex 510, or any other suitable portion of the sensor assembly).

In further embodiments, other methods can be used to transition the barrier component 720 from the first, compact configuration to the second, expanded configuration. For example, in embodiments where the barrier component 720 is comprised at least in part of Nitinol that is manufactured such that its austenite finish (A_{f}) temperature is higher than body temperature, energy (e.g., non-invasive energy) provided by a source internal to or external to the body can be utilized to actuate one or both of the actuating regions 722a, 722b, e.g., by raising the temperature of at least a portion of the barrier component 720 to elicit a geometric change in one or both of the actuating regions 722a, 722b resulting from the shape memory effect (e.g., via inducing a material phase change in the Nitinol material). In further variations, each of the actuating regions 722a, 722b can be moved from the first configuration to the second configuration through the application of one or more forces that deform the corresponding actuating region 722a, 722b into the desired geometry. For example, the delivery system or a separate tool can be used to manipulate the barrier component 720 into the final geometry (e.g., the second configuration) once the barrier component 720 has been removed from the delivery system (e.g., removed from a catheter of the delivery system). Still further embodiments may use additional methods, in addition or as an alternative to the previously described methods, to control the geometry of each of the actuating regions 722a, 722b. In some embodiments, each of the actuating regions 722a, 722b is shape-set into an expanded (second) configuration during a first manufacturing step, and during a second manufacturing step each of the actuating regions 722a, 722b is manipulated into a collapsed delivery (first) configuration and subsequently the attachment feature 800 and/or portions of the sensor assembly's housing 402 are coated with a bioabsorbable material (e.g., polyglycolide (PGA), polylactide (PLA), polylactic-co-glycolic acid (PLGA), etc.) that can hold each of the actuating regions 722a, 722b in the collapsed delivery state (e.g., first configuration). Upon release from a delivery system (not shown), each of the actuating regions 722a, 722b can initially remain in the collapsed state for some period of time (e.g., minutes, hours, days, weeks, etc.) while the bioabsorbable coating is broken down by the body. When the bioabsorbable coating has been sufficiently weakened through biological processes, the superelastic properties of each of the actuating regions 722a, 722b can overcome any remaining retention force, and each of the actuating regions 722a, 722b can automatically move toward its expanded geometry (e.g., the second configuration). Such embodiments with delayed transitions between configurations of the barrier component 720 may be advantageous because the sensor assembly can maintain a relatively slim delivery profile for a period of time even after it has been deployed by the delivery system, and as such if the sensor assembly has been erroneously placed or is otherwise desired to be removed, recapture of the implanted sensor assembly using standard medical tools may be more easily accomplished. Since tissue growth that could interfere with a sensor assembly's measurement components is expected to slowly develop over time, it is expected that the barrier component 720, or any other suitable barrier component configured in accordance with embodiments of the present technology, can be transitioned between the first and second configurations gradually and/or a period of time after implantation without or substantially without tissue growth effecting the performance of the sensor assembly and/or one or more of the measurement components thereof.

In some embodiments, the depth of any aspect of the barrier component 720 (e.g., a height of each of the actuating regions 722a, 722b extends away from the housing 402 of the sensor assembly 400) can be kept at or below a fixed ratio of another dimension of the barrier component 720 (e.g., a length and/or width of each of the actuating regions 722a, 722b, as measured in one or more planes orthogonal to the height). In some embodiments, the depth of each of the actuating regions 722a, 722b can be approximately ⅓ to ½ the distance of the longest non-depth dimension of each of the actuating regions 722a, 722b. Without wishing to be bound by theory, it is believed that actuating regions that are shallow/short relative to the protected area 730 they at least partially encompass can allow for more robust washing in the protected area 730, which can help prevent the protected area 730 of the barrier component 720 from being clogged or filled with tissue and/or other unwanted media. Additionally, as described above, the openings 728a-b can further augment flow of blood in and around the protected area 730 and thereby further reduce the likelihood of the protected area 730 being filled with unwanted media. Although the first and second actuating regions 722a, 722b are described as having generally similar dimensions/geometries in FIGS. 7A-7D, in other embodiments the first and second actuating regions 722a, 722b can have one or more different dimensions and/or geometries. In these and other embodiments, the barrier component 720 can include more actuating regions, such as at least three, four, five, or any other suitable number of actuating regions. Additionally, or alternatively, in some embodiments individual ones of the actuating regions can be transitioned between the first and second configurations selectively and/or independently.

FIGS. 8A and 8B show perspective views of another barrier component 820 in an expanded (e.g., prior to insertion into a delivery catheter, after delivery into the body, etc.) configuration. The barrier component 820 can be generally similar to the barrier component 720 described above with reference to FIGS. 7A-7D, with like numbers (e.g., intermediate region 824 versus the intermediate region 724 of FIGS. 7A-7D) indicating like elements. However, the barrier component 820 includes a single actuating region 822 that partially surrounds or defines a protected area 830. In the illustrated embodiment, for example, the actuating region 822 is positioned on a first side of the pressure-responsive complex 510, and a second, opposite side of the pressure-responsive complex 510 can be open to the external environment. Additionally, or alternatively, the actuating region 822 can be positioned between the pressure responsive complex 510 and the nearest heart wall, e.g., thus serving as a barrier from “bridging” tissues that may grow from the wall to the housing 402 of the sensor assembly 400. Although a barrier as shown may not completely stop or slow tissue formation in the region of a pressure sensor assembly, it may at least partially prevent or delay tissue growth while lowering the risk of blockage, clotting, or other unwanted physiological outcomes due to the presence of fluid, tissues, or other matter within the protected area 830 that can affect the operation of the sensor assembly 400.

FIG. 9 is a perspective view of another barrier component 920 configured in accordance with embodiments of the present technology. The barrier component 920 can be generally similar to the barrier component 720 of FIGS. 7A-7D, with like numbers (e.g., intermediate region 924 versus the intermediate region 724 of FIGS. 7A-D) indicating like elements. However, the barrier component 920 further includes one or more stabilization or coupling regions 932 that can couple the barrier component 920 to a sensor assembly, such as the sensor assembly 400. In the illustrated embodiment, the barrier component 920 includes a first coupling region 932a on a first side of and coupled to the intermediate region 924 and a second coupling region 932b on a second, opposite side of and coupled to the intermediate region 924. In other embodiments, the barrier component 920 can include more or fewer coupling regions 932, such as at least one, three, four, or any other suitable number of coupling regions 932. In these and other embodiments, each of the coupling regions 932 can have any suitable position relative to the intermediate region 924 and/or another region of the barrier component 920. Each of the coupling regions 932 can be configured generally similar or the same as the intermediate region 924, as described previously regarding the intermediate region 724 of FIGS. 7A-7D. The barrier component 920 is expected to exhibit at least some or all of the advantages discussed previously regarding FIGS. 7A-7D. Additionally, it is expected that the one or more coupling regions 932 can further improve the coupling and/or stability of the barrier component 920 relative to a sensor assembly.

### Examples

Several aspects of the present technology are set forth in the following examples:

1. A sensor device for an implantable medical device, the sensor device comprising: a housing, wherein the housing includes a diaphragm portion;
a sensor measurement component positioned within the housing and at least partially aligned with the diaphragm portion, wherein, when the medical device is implanted within a patient, the sensor measurement component is configured to measure one or more physiological parameters of the patient; and
a coupling element positioned between the diaphragm portion and the sensor measurement component and at least partially covering or encapsulating the sensor measurement component, wherein the coupling element is composed of a solid elastomeric material.

2. The sensor device of example 1 wherein the diaphragm portion is configured to transmit the one or more physiological parameters to the measurement component.

3. The sensor device of example 1 or example 2 wherein the coupling element has an end portion, and wherein the diaphragm portion at least partially contacts the end portion.

4. The sensor device of example 3 wherein the diaphragm portion has a first geometry and the end portion has a second geometry, and wherein the first geometry corresponds with the second geometry.

5. The sensor device of any one of examples 1˜4 wherein the diaphragm portion has a first thickness and a portion of the housing surrounding the diaphragm portion has a second thickness greater than the first thickness.

6. The sensor device of any one of examples 1-5 wherein at least a portion of a surface area of the housing further includes a pressure-responsive complex, and wherein the pressure-responsive complex includes the diaphragm portion.

7. The sensor device of example 6 wherein the pressure-responsive complex further includes one or more pressure-sensitive regions.

8. The sensor device of example 6 or example 7 wherein the pressure-responsive complex extends at least partially along an axis parallel to a longitudinal axis of the housing.

9. The sensor device of example 6 or example 7 wherein the pressure-responsive complex extends at least partially radially around the housing in a direction approximately perpendicular to a longitudinal axis of the housing.

10. The sensor device of any one of examples 6-9 wherein the pressure-responsive complex includes at least 5% of a surface area of the housing.

11. The sensor device of any one of examples 6-9 wherein the pressure-responsive complex includes at least 50% of a surface area of the housing.

12. The sensor device of any one of examples 6-11 wherein the pressure-responsive complex has a same thickness as the housing.

13. The sensor device of any one of examples 6-12, further comprising a barrier component proximate to the pressure responsive complex, and wherein the barrier component is configured to at inhibit or otherwise limit host tissue from forming tissues bridges with at least a portion of the pressure-responsive complex.

14. The sensor device of example 13 wherein the barrier component is positioned to function as a strain relief mechanism that at least partially limits transmittal of forces from native tissues to at least a portion of the pressure-responsive complex.

15. The sensor device of example 13 wherein the barrier component defines an outer perimeter or boundary that at least partially surrounds the pressure-responsive complex.

16. The sensor device of any one of examples 13-15 wherein the barrier component is composed of Nitinol.

17. A barrier component for an implantable medical device, the barrier component comprising:
an actuating region having a first end portion, a second end portion opposite to and spaced apart from the first end portion, and a surface between the first end portion the second end portion; and
a support region coupled to the actuating region between the first end portion and the second end portion and opposite the surface,
wherein the actuating region is configured to be transitionable between—
   a first configuration in which the first end portion faces a first direction, the second end portion faces a second direction opposite the first direction, and the surface is generally linear, and
   a second configuration in which the first end portion and the second end portion face a same direction and the surface is generally curved.

18. The barrier component of example 17 wherein the implantable medical device includes a sensor assembly, and wherein the barrier component further comprises an opening configured to at least partially releasably receive the sensor assembly.

19. The barrier component of example 18 wherein the support region is releasably couplable to the sensor assembly when the sensor assembly is received by the opening of the barrier component.

20. The barrier component of example 18 or example 19 wherein the actuating region is at least partially aligned with a portion of the sensor assembly.

21. The barrier component of example 20 wherein the portion of the sensor assembly includes a pressure-responsive complex of the sensor assembly.

22. The barrier component of example 21 wherein:
in the first configuration, the actuating region at least partially covers the portion of the sensor assembly, and
in the second configuration, the actuating region defines a barrier extending at least partially around the portion of the sensor assembly.

23. The barrier component of any one of examples 17-22 wherein the actuating region is composed of a shape-memory material.

24. The barrier component of any one of examples 17-23 wherein the actuating region is composed of Nitinol.

25. The barrier component of any one of examples 17-24 wherein the actuating region is configured to automatically transition between the first configuration and the second configuration.

26. The barrier component of any one of examples 17-24 wherein the actuating region is configured to transition between the first configuration and the second configuration in response to non-invasive laser energy.

27. The barrier component of any one of examples 17-26 wherein the first end portion includes a first opening and the second end portion includes a second opening.

28. The barrier component of any one of examples 17-27 wherein the surface is a first surface, and wherein the barrier component further comprises:
a second surface opposite the first surface and between the first end portion and the support region; and
a third surface opposite the first surface and the between the second end portion and the support surface;
wherein the second surface and third surface each has a curvature that corresponds to sensor assembly when the barrier component is in the second configuration.

29. The barrier component of any one of examples 17-27 wherein the actuating region is a first actuating region and the surface is a first surface, and wherein the barrier component further comprises a second actuating region, wherein:
the second actuating region includes a third end portion, a fourth end portion opposite and spaced apart from the third end portion, and a second surface extending between the third end portion and the fourth end portion;
the support region is coupled to the second actuating region between the third end portion and the fourth end portion and opposite the second surface; and
the second actuating region is transitionable between—
   a first configuration in which the third end portion faces the first direction, the second end portion faces the second direction, and the second surface is generally parallel to the first surface, and
   a second configuration in which the third end portion and the fourth end portion face a same direction and the second surface is generally curved.

30. The barrier component of example 29 wherein, in the second configuration:
the third end portion of the second actuating region faces toward the first end portion of the first actuating region, and
the fourth end portion of the second actuating region faces toward the second end portion of the first actuating region.

31. The barrier component of example 29 or example 30 wherein the first end portion includes a first recess, the second end portion includes a second recess, the third end portion includes a third recess, and the fourth end portion includes a fourth recess.

32. The barrier component of example 31 wherein, in the second configuration, the first recess is positioned to align with the third recess to form a first opening and the second recess is positioned to aligned with the fourth recess to form a second opening.

33. The barrier component of any one of examples 17-32 wherein the support region is configured to releasably couple the barrier component to the implantable medical device.

34 The barrier component of any one of examples 17-33, further comprising one or more coupling regions, wherein each of the coupling regions are configured to releasably couple the barrier component to the implantable medical device.

35. The barrier component of example 34 wherein the one or more coupling regions comprise:
a first coupling region positioned on a first side of the support region; and
a second coupling region position on a second side of the support region and opposite the first coupling region.

36. A sensor device for an implantable medical device, the sensor device comprising:
a housing, wherein the housing includes a pressure-responsive complex portion, and wherein the pressure-responsive complex portion includes at least 5% of a surface area of the housing;
a sensor measurement component positioned within the housing and aligned with the pressure-responsive complex portion, wherein, when the medical device is implanted within a patient, the sensor measurement component is configured to measure one or more physiological parameters of the patient; and
a coupling element positioned between the pressure-responsive complex portion and the sensor measurement component and at least partially covering or encapsulating the sensor measurement component, wherein the coupling element is composed of a solid elastomeric material.

37. The sensor device of example 36 wherein the pressure-responsive complex portion extends at least partially along an axis parallel to a longitudinal axis of the housing.

38. The sensor device of example 36 wherein the pressure-responsive complex portion extends at least partially radially around the housing in a direction approximately perpendicular to a longitudinal axis of the housing.

39. The sensor device of any one of examples 36-38 wherein the pressure-responsive complex portion has a same thickness as the housing.

40. The sensor device of any one of examples 36-39 wherein the pressure-responsive complex portion is configured to transmit the one or more physiological parameters to the sensor measurement component.

41. A sensor device for an implantable medical device, the sensor device comprising:
a housing, wherein the housing includes a pressure-responsive complex region, and wherein the pressure-responsive complex region has a same thickness as the housing;
a sensor measurement component positioned within the housing and aligned with the pressure-responsive complex region, wherein, when the medical device is implanted within a patient, the sensor measurement component is configured to measure one or more physiological parameters of the patient; and
a coupling element positioned between the pressure-responsive complex region and the sensor measurement component and at least partially covering or encapsulating the sensor measurement component, wherein the coupling element is composed of a solid elastomeric material.

42. The sensor device of example 41 wherein the pressure-responsive complex region extends at least partially along an axis parallel to a longitudinal axis of the housing.

43. The sensor device of example 41 wherein the pressure-responsive complex region extends at least partially radially around the housing in a direction approximately perpendicular to a longitudinal axis of the housing.

44. The sensor device of any one of examples 41-43 wherein the pressure-responsive complex region includes at least 5% of a surface area of the housing.

45. The sensor device of any one of examples 41-43 wherein the pressure-responsive complex region includes at least 50% of a surface area of the housing.

46. The sensor device of any one of examples 41-45 wherein the pressure-responsive complex region is configured to transmit the one or more physiological parameters to the sensor measurement component.

47. A method of manufacturing a pressure sensor device configured for use in an implantable medical device, the method comprising:
providing a substrate and a tool, wherein the tool has an end portion including a pattern;
positioning the tool such that the end portion at least partially contacts a first side of substrate;
forming a pressure sensitive diaphragm in the substrate, wherein forming the diaphragm includes applying a force to the tool in the direction of the substrate to create a first deformation in the first side of the substrate, wherein the substrate has a first thickness, and the diaphragm has a second thickness less than the first thickness; and
positioning a sensor measurement component proximate to the second side of the substrate, wherein the sensor is communicatively coupled to the diaphragm.

48. The method of example 47 wherein the substrate further includes a second side opposite the first side, and wherein applying a force to the tool further includes creating a second deformation in the second side of the substrate.

49. The method of example 47 or example 48 wherein the pattern is configured to correspond to a predetermined geometry of the diaphragm.

50. The method of any one of examples 47-49 wherein the substrate includes a single sheet of material.

51. The method of any one of examples 47-50 wherein the deformations include one or more regions of a pressure responsive complex, and wherein forming the diaphragm includes forming at least one of the one or more regions.

52. The method of example 51 wherein the one or more regions are concentric.

53. The method of example 51 wherein the one or more regions are corrugated.

54. The method of example 51 wherein each of the one or more regions has a circular, triangular, square, pentagonal, or hexagonal shape.

55. The method of example 51 wherein the one or more regions include one or more curves relative to the first and/or second side of the substrate.

56. The method of any one of examples 47-55 wherein forming the pressure sensitive diaphragm in the substrate comprises forming the diaphragm having the second thickness such that the diaphragm is responsive to pressure changes in an environment proximate the substrate.

57. The method of any one of examples 47-56 wherein the sensor device is a MEMS sensor and, wherein, when the medical device is implanted within a patient, the sensor device is configured to measure a pressure in a body chamber of the patient.

58. The method of any one of examples 47-57 wherein positioning the sensor measurement component includes operably coupling the sensor measurement component to the second side of the substrate.

59. The method of any one of examples 47-58 wherein, when the medical device is implanted within a patient, the sensor device is configured to measure one or more physiological parameters of the patient.

60. The method of any one of examples 47-59, further comprising positioning a coupling material at least partially between the diaphragm and the sensor measurement component.

61. The method of example 60 wherein the coupling material is configured to communicatively couple the diaphragm to the sensor measurement component.

62. The method of example 61 wherein the coupling material is a solid elastomeric material.

63. The method of example 62 wherein the solid elastomeric material is polydimethylsiloxane.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A sensor device for an implantable medical device, the sensor device comprising:
a housing, wherein the housing includes a diaphragm portion;
a sensor measurement component positioned within the housing and at least partially aligned with the diaphragm portion, wherein, when the medical device is implanted within a patient, the sensor measurement component is configured to measure one or more physiological parameters of the patient; and
a coupling element positioned between the diaphragm portion and the sensor measurement component and at least partially covering or encapsulating the sensor measurement component, wherein the coupling element is composed of a solid elastomeric material.

2. The sensor device of claim 1 wherein the diaphragm portion is configured to transmit the one or more physiological parameters to the measurement component.

3. The sensor device of claim 1 wherein the coupling element has an end portion, and wherein the diaphragm portion at least partially contacts the end portion.

4. The sensor device of claim 3 wherein the diaphragm portion has a first geometry and the end portion has a second geometry, and wherein the first geometry corresponds with the second geometry.

5. The sensor device of claim 1 wherein the diaphragm portion has a first thickness and a portion of the housing surrounding the diaphragm portion has a second thickness greater than the first thickness.

6. The sensor device of claim 1 wherein at least a portion of a surface area of the housing further includes a pressure-responsive complex, and wherein the pressure-responsive complex includes the diaphragm portion.

7. The sensor device of claim 6 wherein the pressure-responsive complex further includes one or more pressure-sensitive regions.

8. The sensor device of claim 6 wherein the pressure-responsive complex extends at least partially along an axis parallel to a longitudinal axis of the housing.

9. The sensor device of claim 6 wherein the pressure-responsive complex extends at least partially radially around the housing in a direction approximately perpendicular to a longitudinal axis of the housing.

10. The sensor device of claim 6 wherein the pressure-responsive complex includes at least 5% of a surface area of the housing.

11. The sensor device of claim 6 wherein the pressure-responsive complex includes at least 50% of a surface area of the housing.

12. The sensor device of claim 6 wherein the pressure-responsive complex has a same thickness as the housing.

13. The sensor device of claim 6, further comprising a barrier component proximate to the pressure responsive complex, and wherein the barrier component is configured to at inhibit or otherwise limit host tissue from forming tissues bridges with at least a portion of the pressure-responsive complex.

14. The sensor device of claim 13 wherein the barrier component is positioned to function as a strain relief mechanism that at least partially limits transmittal of forces from native tissues to at least a portion of the pressure-responsive complex.

15. The sensor device of claim 13 wherein the barrier component defines an outer perimeter or boundary that at least partially surrounds the pressure-responsive complex.

16. The sensor device of claim 13 wherein the barrier component is composed of Nitinol.

17. A barrier component for an implantable medical device, the barrier component comprising:
an actuating region having a first end portion, a second end portion opposite to and spaced apart from the first end portion, and a surface between the first end portion the second end portion; and
a support region coupled to the actuating region between the first end portion and the second end portion and opposite the surface,
wherein the actuating region is configured to be transitionable between—
a first configuration in which the first end portion faces a first direction, the second end portion faces a second direction opposite the first direction, and the surface is generally linear, and
a second configuration in which the first end portion and the second end portion face a same direction and the surface is generally curved.

18. The barrier component of claim 17 wherein the implantable medical device includes a sensor assembly, and wherein the barrier component further comprises an opening configured to at least partially releasably receive the sensor assembly.

19. The barrier component of claim 18 wherein the support region is releasably couplable to the sensor assembly when the sensor assembly is received by the opening of the barrier component.

20. The barrier component of claim 18 wherein the actuating region is at least partially aligned with a portion of the sensor assembly.

21. The barrier component of claim 20 wherein the portion of the sensor assembly includes a pressure-responsive complex of the sensor assembly.

22. The barrier component of claim 20 wherein:
in the first configuration, the actuating region at least partially covers the portion of the sensor assembly, and
in the second configuration, the actuating region defines a barrier extending at least partially around the portion of the sensor assembly.

23. The barrier component of claim 17 wherein the actuating region is composed of a shape-memory material.

24. The barrier component of claim 17 wherein the actuating region is composed of Nitinol.

25. The barrier component of claim 17 wherein the actuating region is configured to automatically transition between the first configuration and the second configuration.

26. The barrier component of claim 17 wherein the actuating region is configured to transition between the first configuration and the second configuration in response to non-invasive laser energy.

27. The barrier component of claim 17 wherein the first end portion includes a first opening and the second end portion includes a second opening.

28. The barrier component of claim 17 wherein the surface is a first surface, and wherein the barrier component further comprises:
a second surface opposite the first surface and between the first end portion and the support region; and
a third surface opposite the first surface and the between the second end portion and the support surface;
wherein the second surface and third surface each has a curvature that corresponds to sensor assembly when the barrier component is in the second configuration.

29. The barrier component of claim 17 wherein the actuating region is a first actuating region and the surface is a first surface, and wherein the barrier component further comprises a second actuating region, wherein:
the second actuating region includes a third end portion, a fourth end portion opposite and spaced apart from the third end portion, and a second surface extending between the third end portion and the fourth end portion;
the support region is coupled to the second actuating region between the third end portion and the fourth end portion and opposite the second surface; and
the second actuating region is transitionable between—
a first configuration in which the third end portion faces the first direction, the second end portion faces the second direction, and the second surface is generally parallel to the first surface, and
a second configuration in which the third end portion and the fourth end portion face a same direction and the second surface is generally curved.

30. The barrier component of claim 29 wherein, in the second configuration:
the third end portion of the second actuating region faces toward the first end portion of the first actuating region, and
the fourth end portion of the second actuating region faces toward the second end portion of the first actuating region.

31. The barrier component of claim 29 wherein the first end portion includes a first recess, the second end portion includes a second recess, the third end portion includes a third recess, and the fourth end portion includes a fourth recess.

32. The barrier component of claim 31 wherein, in the second configuration, the first recess is positioned to align with the third recess to form a first opening and the second recess is positioned to aligned with the fourth recess to form a second opening.

33. The barrier component of claim 17 wherein the support region is configured to releasably couple the barrier component to the implantable medical device.

34. The barrier component of claim 17, further comprising one or more coupling regions, wherein each of the coupling regions are configured to releasably couple the barrier component to the implantable medical device.

35. The barrier component of claim 34 wherein the one or more coupling regions comprise:
a first coupling region positioned on a first side of the support region; and
a second coupling region position on a second side of the support region and opposite the first coupling region.

36. A sensor device for an implantable medical device, the sensor device comprising:
a housing, wherein the housing includes a pressure-responsive complex portion, and wherein the pressure-responsive complex portion includes at least 5% of a surface area of the housing;
a sensor measurement component positioned within the housing and aligned with the pressure-responsive complex portion, wherein, when the medical device is implanted within a patient, the sensor measurement component is configured to measure one or more physiological parameters of the patient; and
a coupling element positioned between the pressure-responsive complex portion and the sensor measurement component and at least partially covering or encapsulating the sensor measurement component, wherein the coupling element is composed of a solid elastomeric material.

37. The sensor device of claim 36 wherein the pressure-responsive complex portion extends at least partially along an axis parallel to a longitudinal axis of the housing.

38. The sensor device of claim 36 wherein the pressure-responsive complex portion extends at least partially radially around the housing in a direction approximately perpendicular to a longitudinal axis of the housing.

39. The sensor device of claim 36 wherein the pressure-responsive complex portion has a same thickness as the housing.

40. The sensor device of claim 36 wherein the pressure-responsive complex portion is configured to transmit the one or more physiological parameters to the sensor measurement component.

41. A sensor device for an implantable medical device, the sensor device comprising:
a housing, wherein the housing includes a pressure-responsive complex region, and wherein the pressure-responsive complex region has a same thickness as the housing;
a sensor measurement component positioned within the housing and aligned with the pressure-responsive complex region, wherein, when the medical device is implanted within a patient, the sensor measurement component is configured to measure one or more physiological parameters of the patient; and
a coupling element positioned between the pressure-responsive complex region and the sensor measurement component and at least partially covering or encapsulating the sensor measurement component, wherein the coupling element is composed of a solid elastomeric material.

42. The sensor device of claim 41 wherein the pressure-responsive complex region extends at least partially along an axis parallel to a longitudinal axis of the housing.

43. The sensor device of claim 41 wherein the pressure-responsive complex region extends at least partially radially around the housing in a direction approximately perpendicular to a longitudinal axis of the housing.

44. The sensor device of claim 41 wherein the pressure-responsive complex region includes at least 5% of a surface area of the housing.

45. The sensor device of claim 41 wherein the pressure-responsive complex region includes at least 50% of a surface area of the housing.

46. The sensor device of claim 41 wherein the pressure-responsive complex region is configured to transmit the one or more physiological parameters to the sensor measurement component.

47. A method of manufacturing a pressure sensor device configured for use in an implantable medical device, the method comprising:
providing a substrate and a tool, wherein the tool has an end portion including a pattern;
positioning the tool such that the end portion at least partially contacts a first side of substrate;
forming a pressure sensitive diaphragm in the substrate, wherein forming the diaphragm includes applying a force to the tool in the direction of the substrate to create a first deformation in the first side of the substrate, wherein the substrate has a first thickness, and the diaphragm has a second thickness less than the first thickness; and
positioning a sensor measurement component proximate to the second side of the substrate, wherein the sensor is communicatively coupled to the diaphragm.

48. The method of claim 47 wherein the substrate further includes a second side opposite the first side, and wherein applying a force to the tool further includes creating a second deformation in the second side of the substrate.

49. The method of claim 47 wherein the pattern is configured to correspond to a predetermined geometry of the diaphragm.

50. The method of claim 47 wherein the substrate includes a single sheet of material.

51. The method of claim 47 wherein the deformations include one or more regions of a pressure responsive complex, and wherein forming the diaphragm includes forming at least one of the one or more regions.

52. The method of claim 51 wherein the one or more regions are concentric.

53. The method of claim 51 wherein the one or more regions are corrugated.

54. The method of claim 51 wherein each of the one or more regions has a circular, triangular, square, pentagonal, or hexagonal shape.

55. The method of claim 51 wherein the one or more regions include one or more curves relative to the first and/or second side of the substrate.

56. The method of claim 47 wherein forming the pressure sensitive diaphragm in the substrate comprises forming the diaphragm having the second thickness such that the diaphragm is responsive to pressure changes in an environment proximate the substrate.

57. The method of claim 47 wherein the sensor device is a MEMS sensor and, wherein, when the medical device is implanted within a patient, the sensor device is configured to measure a pressure in a body chamber of the patient.

58. The method of claim 47 wherein positioning the sensor measurement component includes operably coupling the sensor measurement component to the second side of the substrate.

59. The method of claim 47 wherein, when the medical device is implanted within a patient, the sensor device is configured to measure one or more physiological parameters of the patient.

60. The method of claim 47, further comprising positioning a coupling material at least partially between the diaphragm and the sensor measurement component.

61. The method of claim 60 wherein the coupling material is configured to communicatively couple the diaphragm to the sensor measurement component.

62. The method of claim 61 wherein the coupling material is a solid elastomeric material.

63. The method of claim 62 wherein the solid elastomeric material is polydimethylsiloxane.
